(19) United States
(12) Patent Application Publication
(10) Pub. No.: US 2026/0216091 A1
(43) Pub. Date: Jul. 30, 2026

(54) NANOPARTICLE COMPOSITIONS AND USES THEREOF

(71) Applicants: OP-T LLC, Aurora, CO (US); The Regents of the University of Colorado, a body corporate, Denver, CO (US)

(72) Inventors: David H. Wagner, JR., Denver, CO (US); Martin G. Yussman, Denver, CO (US); Gisela M. Vaitaitis, Centennial, CO (US); Charles W. Henry, Denver, CO (US)

(21) Appl. No.: 19/280,907

(22) Filed: Jul. 25, 2025

Related U.S. Application Data
(63) Continuation of application No. 17/320,907, filed on May 14, 2021, now abandoned.
(60) Provisional application No. 63/025,132, filed on May 14, 2020.

(51) Int. Cl. A61K 9/51; A61K 38/08; A61K 38/10; A61K 38/17; A61K 47/69; C07K 14/705

(52) U.S. Cl. CPC A61K 9/5153; A61K 38/08; A61K 38/10; A61K 38/179; A61K 47/6929; C07K 14/70575; C07K 14/70578

(57) ABSTRACT

The present developments provide methods and compositions for treating and/or preventing autoimmune diseases. In certain aspects, the present disclosure relates to the use of short peptides loaded inside of nanoparticle nanospheres, nanocapsules, or PEGylated nanoparticles. Additionally, peptide-metal nanoparticle drug conjugates are described and disclosed. In some embodiments, these nanoparticles, whether polymer based or metal-conjugated, when linked or coupled to bioactive peptides provided herein, may be capable of interacting with CD40 proteins or CD40 complexes, and thereby may interfere with the ability of CD40 to interact with CD154. The present disclosure also relates to the use of such nanoparticle-peptide conjugates in reducing the inflammatory response, and in particular, the autoimmune inflammatory response. The present disclosure also relates to the use of such short peptides to prevent or reverse autoimmune disease, in particular in type 1 diabetes and multiple sclerosis, in individuals suffering from such diseases.

### RELATED APPLICATIONS

This application is a Continuation of U.S. patent application Ser. No. 17/320,907, filed May 14, 2021, which claims priority to U.S. Provisional Application No. 63/025,132, filed May 14, 2020, the content of each of which is hereby incorporated herein by reference in its entirety.

### SEQUENCE LISTING

This application contains a Sequence Listing which has been submitted electronically in XML format. The Sequence Listing XML is incorporated herein by reference. Said XML file, created on Apr. 7, 2026, is named OPB-01102_SL.xml and is 46,187 bytes in size.

### BACKGROUND

Autoimmune related diseases are on the rise worldwide at a greater extent than genetics alone might predict. Current research postulates that autoimmune disease may be impacted by both genetic and environmental factors. These environmental factors that may amplify autoimmunity may include but are not limited to lifestyle, e.g. a poor diet and lack of exercise, air and water pollution, stress, tobacco and alcohol use, etc. Although the exact environmental mechanisms that induces or propels autoimmune disease have not been pinpointed, the research is clear that aseptic inflammation drives the development of autoimmunity.

One molecular dyad central to aseptic inflammation is the CD40-CD154 receptor-ligand pair. CD40 ligand (CD154) interacts with CD40 on numerous types of cells resulting in cellular signaling that implicate various consequences and downstream effects. In the instance that the CD40 bearing cell is an immune cell, such as bone marrow, stem cells, thymus cells, T-lymphocytes, B-cells, cytotoxic T-cells, helper T-cells, plasma cells, neutrophils, monocytes, red blood cells, platelets, and dendritic cells, the downstream consequence may often activate inflammatory signals and result in the production of inflammatory cytokines. This activation of inflammatory signals and production of inflammatory cytokines may be beneficial if the signaling cascade was induced by the presence of a pathogen that needs to be neutralized; however, in the case of autoimmunity, that is, where the immune system attacks self-tissues without the presence of a pathogen, CD40 signals may be detrimental and drive the autoimmunity.

Autoimmune diseases that are impacted by CD40 signals include, but are not limited to, Type 1 Diabetes (TID), Multiple Sclerosis (MS), Rheumatoid Arthritis (RA), Systemic Lupus Erythematosus (SLE), chronic obstructive pulmonary disease (COPD), acute respiratory distress syndrome (ARDS), and cardiovascular disease (CVD). To combat autoimmune disease, CD40 has been examined and targeted by researchers for several decades. Numerous efforts have been made to develop pharmaceutical agents to target CD40 and the focus of many efforts has been the use of monoclonal antibodies. In at least some cases, however, targeting CD40 itself has proven challenging and unfeasible because CD40 antibodies are agonistic and tend to enhance CD40 signaling, rather than prevent it. Thus, practitioners targeted CD154 instead and have achieved some moderate levels of success.

For example, numerous studies in mouse models have demonstrated that monoclonal antibodies to CD154 may be utilized to prevent several of the various autoimmune diseases; however, in clinical trials for use in humans, patients in some instances experienced severe adverse events such as life-threatening embolic reactions. Consequently, sponsors halted the studies and further research using the specific antibodies.

Thus, pursuits to find other ways of altering or blocking CD40 signals is still ongoing on multiple fronts. For example, in one study researchers screened random peptides for their ability to block CD40 in-vitro, but because these peptides were random results from in-vivo may have unforeseen side effects as the peptides may have bound to other off-target binding sites. Additionally, in some instances these studies may have required very high concentrations. In other efforts, investigators employed small organic molecules, and while some of these small organic molecules may block CD40 in-vitro, they have undesirable and/or toxic side effects and may lose their efficacy in protein rich medium.

Human TID patients administer insulin subcutaneously several times a day. This often means that the subject must carry with them the supplies, i.e. insulin and syringes, needed to administer the insulin doses. Additionally, administration of insulin requires alternating injection sites so that localized irritation does not occur.

Likewise, in some human MS patients, the drug Copaxone® (glatiramer acetate) (Teva Pharmaceutical Industries Ltd., Jerusalem, Israel), is administered daily and in some instances irritation at the injection site occurs. Several other pharmaceutical drugs used by MS patients such as Avonex® (Biogen, Inc., Cambridge, MA, USA), Betaseron® (Schering Aktiengesellschaft Corp., Germany), Extavia® (Novartis AG, Basel, Switzerland), Plegridy® (Biogen, Inc., Cambridge, MA, USA), and Rebif® (Ares Trading SA, Aubonne, Switzerland), are also provided as injectable formularies and thus may also cause the patient to experience localized irritation. Several of these MS treatment regimens provide that the drug is continuously administered, such as once a day or more injections, so that the patient is treated at all times of day, even when they do not experience symptoms. This may be undesirable since many of the available treatments can have significant side effects and thus the patient may suffer those side effects unnecessarily. Moreover, some available MS treatments utilize antibodies or monoclonal antibodies that may also have undesirable side effects, such as cytokine release syndrome and antigenicity, both of which can be quite severe and, in some instances, life threatening. Another issue for injectable insulin and several MS treatments is that the therapies target the symptoms rather than the underlying drivers of autoimmune disease.

Thus, alternative compositions and methods for treatment and prevention of diseases implicated and affected by the CD40-CD154 inflammatory dyad may be desirable.

### SUMMARY

In certain aspects, provided herein are novel methods and compositions for modulating, affecting, and/or regulating inflammation, particularly autoimmune inflammation, that may be mediated and/or influenced by CD40-CD154 mediated mechanisms. One implementation includes a novel composition of matter including nanoparticles (NPs) that can be used in the delivery of therapeutic agents via several routes of administration. One implementation utilizes poly(lactic-co-glycolic acid) (PLGA), which is a biodegradable polymer. Different forms of PLGA can be generated depending on the ratio of the monomers, lactic acid and glycolic acid that is used. In one implementation, a PLGA 50:50 may be utilized. Here, “50:50” denotes a copolymer that is composed of 50% lactic acid and 50% glycolic acid. PLGA may be utilized as it may provide therapeutic results with only minimal systemic toxicity. The degradation time of PLGA may vary but is often dependent on the copolymer ratio as well as the molecular weight. PLGA NPs may provide the added benefit of providing a mechanism of delivering a therapeutic peptide intracellularly, instead of simply delivering the peptide extracellularly. PLGA NPs may enter cells via pinocytosis (invagination of the cell membrane) and clathrin-mediated endocytosis (a vesicle formed and coated by the protein clathrin). Once taken up by the cell, the PLGA NPs may be released into the cytoplasm in a short period of time, i.e. 10 minutes.

In one aspect disclosed herein, the PLGA NPs provided herein may be formulated with tunable sizes, ranging from nano- to micrometer. This may be accomplished by using D-α-Tocopheryl polyethylene glycol 1000 succinate (simply TPGS or Vitamin E TPGS) in an emulsion technique with ultrasonication in non-miscible solvents followed by evaporation and ultracentrifugation.

In certain aspects, provided herein are improved routes of administration, using nanoparticle technology. In some embodiments, these developments are based on the knowledge that interaction of CD40-ligand (CD154 protein) with CD40 protein expressed on T-cells (Th40 cells) that may be important in the development of autoimmune disease. The development may also be based on the elucidation of the critical residues in CD40 and CD154 that are important for this interaction. The present developments may also relate to blocking or altering of the interaction between a CD40 complex and a CD154 protein through the use of small peptides that interact with the CD40 complex at a site where the CD154 protein would normally bind. The present developments may also relate to using such peptides to reduce the level of Th40 cells, thereby reducing the severity of disease.

Thus, the developments and implementations disclosed herein may include compositions of matter and methods of treating and/or reducing the incidence of type 1 diabetes, multiple sclerosis, rheumatoid arthritis, systemic lupus erythematosus, chronic obstructive pulmonary disease, atherosclerosis, cytokine release syndrome, COVID-19, long-COVID-19, and/or acute respiratory distress syndrome. In some embodiments, these methods include administering to a subject in need of a prophylactic or treatment an effective amount of a peptide to inhibit, interfere, or affect the binding between proteins associated with CD40 complex, including but not limited to CD154. In these methods, the diseases or disorders may include one or more of type 1 diabetes, multiple sclerosis, rheumatoid arthritis, systemic lupus erythematosus, chronic obstructive pulmonary disease, atherosclerosis, acute respiratory distress syndrome, hyperglycemia, and/or any another condition or indication as that implicates autoimmune related inflammation, including but not limited to Hashimoto's thyroiditis and Graves' disease, which affect the thyroid gland; pernicious anemia, which affects the blood; Addison's disease, which affects the adrenal glands; chronic active hepatitis, which affects the liver; and myasthenia gravis which affects the muscle.

In some embodiments, the developments are based on the knowledge that interaction of CD40-ligand (CD154 protein) with CD40 protein expressed on T-cells (Th40 cells) and other immune cells and is an important pathway in inflammatory disorders. In some embodiments, this development is facilitated through the use of unique peptide KGYY15, with a sequence derived from the CD154 protein. CD154 (a member of the TNF super family) is the ligand for CD40 and the mouse CD154-derived sequence VLQWAKKGYYTMKSN spans a region with CD40-interacting amino acids. Thus, in some embodiments, the developments provided herein may also be based on the elucidation of the critical residues in CD40 and CD154 that are important for this interaction. In some embodiments, the present developments relate to blocking, altering, or modifying the interaction between a CD40 protein or CD40 complex and a CD154 protein using small peptides. In certain embodiments, the present developments may also relate to using such peptides to reduce the level of CD40 presenting cells, thereby reducing the severity of disease.

One implementation of the present developments may include a peptide that interacts with CD4+ T cells in such a manner as to induce tolerance. In this development, the initiation and progression of tolerance may result in the establishment or re-establishment of homeostatic balance within in the subject.

Another implementation may be a peptide that interacts with CD40 on antigen presenting cells (APCs) in such a manner as to induce tolerance on both APCs and subsequent T cells leading to homeostatic balance. In this development, peptides that target CD40 may regulate antigen-presenting cells to create tolerance, wherein, B-cell auto-antibody production may be affected, B-cell antigen presentation may be affected, and macrophage pro-inflammatory pathways may be affected.

Yet another implementation of the current developments includes a composition of matter of a peptide and nanoparticle and method for targeting CD40 on beta cells which may reduce the beta cell auto-antigen production. In this embodiment, the reduction of beta cell auto-antigen production may alleviate the need for immune tolerance. Targeting CD40 on beta cells can also restore beta cell insulin production and/or reduce or prevent engagement of CD40 on beta cells.

Yet another implementation of the current developments includes a NP that may target antigen presenting cells (APC), macrophages, and dendritic cells. In such embodiments, the peptide drug may be delivered to the APC and may alter the function of the APC. Macrophages produce inflammatory cytokines including IL-la, IL-1b, IL-6, TNFα, and IL-10. Accordingly, one feature may be that the NPs, nanocapsules, or site targeting NPs or nanocapsules, may be used to control innate inflammation in that way.

Yet another implementation of the current developments may include novel methods for detecting Th40 cells including using nanoparticle technology to facilitate in the detection. Thus, in some embodiments, the present developments may also relate to at least two additional delivery mechanisms to deliver peptides: hollow poly(lactic-co-glycolic acid) (PLGA) nanoparticles (termed PLGA NPs) and functionalized gold nanoparticles (Au NPs) with peptides tethered or conjugated to the surface. It should be noted that the gold nanoparticles may in some instances be replaced or substituted with another metal that may provide the desirable features and similar functionality as it relates to the delivery of peptides and stability of a nanoparticle drug conjugate.

Thus, one implementation of the present developments is an improved delivery composition of the peptide wherein the peptide is loaded into hollow poly(lactic-co-glycolic acid) nanoparticles. And another implementation includes an improved delivery composition of the peptide wherein the peptide is tethered to the surface of functionalized gold nanoparticles.

In some embodiments, the inhibitor peptide compositions of matter and methods of use thereof may be a peptide that binds to CD40, or one or more molecules that may be known in the art to be associated, complexed with, coupled to, or present with CD40. In one aspect, the inhibitor peptides provided herein may disrupt, affect, or modify the interaction of CD40 complex with CD154. In one embodiment, the peptides provided herein may also reduce the number of Th40 cells in the subject. The inhibitor peptides provided herein may include an amino acid sequence selected from the group of SEQ ID NO: 3, SEQ ID NO: 4, SEQ ID NO: 27, SEQ ID NO: 28, SEQ ID NO: 29, and SEQ ID NO: 30. In certain embodiments, the bioactive peptides provided herein may additionally contain the amino acid sequence of SEQ ID: 3. These inhibitor peptides may include a modification selected from phosphorylation and glycosylation, and/or may be linked to a polyethylene glycol (PEG) molecule, and/or may be linked to one or more domains of an Fc region of human IgG immunoglobin, and/or may be linked to an epitope tag polypeptide comprising between 6 and 50 amino acid residues. Additionally, the inhibitor peptides provided herein may also include modifications of termini such as acetylation and amidation. Additionally, peptides provided herein may include several salt forms including hydrochloride salts, acetate salts, TFA salts, and sodium chloride salts.

In some embodiments, the present development also relates to two delivery mechanisms to deliver peptides through: functionalized gold nanoparticles (Au NPs) with peptides tethered to the surface and hollow poly(lactic-co-glycolic acid) (PLGA) nanoparticles (termed PLGA NPs).

Moreover, in additional and alternative implementations, the therapeutic peptide provided herein, may be administered to a subject in combination with a therapeutically effective amount of one or more compounds including: (1) N-acetylcysteine (NAC), (2) ergothioneine (ERGO), (3) alpha-lipoic acid, (4) a CD40+ T cell reducing or inactivating agent, and/or (5) a hypoglycemic agent. Administration is described in more detail below; however, the developments provided herein contemplate and include IV bolus, oral, infusion, subcutaneous, intramuscular, rectal, and/or vaginal routes of administration. Moreover, the therapeutic peptide may also be administered to a subject in combination with PLGA NPs and/or Au NPs and one or more of N-acetylcysteine (NAC), ergothioneine (ERGO), alpha-lipoic acid, a CD40+ T cell reducing or inactivating agent, and/or a hypoglycemic agent.

This Summary is neither intended nor should it be construed as being representative of the full extent and scope of the present disclosure. Moreover, references made herein to “the present disclosure” and/or “present implementation” or aspects or embodiments thereof, should be understood to mean certain implementations of the present disclosure and should not necessarily be construed as limiting all implementations to a particular and/or description. These as well as other alternative and/or additional aspects are exemplified in a number of illustrated alternative and/or additional implementations and applications, some of which are shown in the figures and characterized in the claims section that follows. The above summary and the detailed description below are not intended to describe each illustrated embodiment or every implementation of the present developments, nor to provide any limitation on the claims or scope of protection herein set forth below. Additional features, versions, characteristics and aspects of the present disclosure may become more readily apparent from the Detailed Description, particularly when taken together with the drawings.

### BRIEF DESCRIPTION OF THE DRAWINGS

The drawings include:

FIG. 1A and FIG. 1B provide a sequence listing and a graph of the effect of various exemplary peptides on the development of diabetes in NOD mice.

FIG. 2A, FIG. 2B, FIG. 2C, and FIG. 2D provide graphs of blood glucose levels of different treatments, bar graphs of islet score percentages and insulin granule scores.

FIG. 3A and FIG. 3B provide graphs of percent diabetic mice compared to RGD peptide, scrambled, and treated mice and a graph of percent diabetic when other peptides with substitutions of certain amino acids.

FIG. 4A, FIG. 4B, and FIG. 4C provide western blots, histogram, and graphs of KGYY 15 peptide.

FIG. 5 provides a graph of EAE score of KGYY₆ treated mice versus control.

FIG. 6 provides a graph comparing EAE scores of mice treated under different conditions.

FIG. 7 provides a graph comparing pre-treated mice and the effect of early and later boosts on the progression of EAE.

FIG. 8A and FIG. 8B provide graphs regarding the percentage of peptides binding to CD3+ T cells and percentage of peptides binding to antigen presenting cells.

FIG. 9 provides a diagram of the hydrolysis of PLGA.

FIG. 10 provides a schematic of nanoparticle internalization in cells.

FIG. 11A, FIG. 11B, FIG. 11C, FIG. 11D, FIG. 11E, FIG. 11F, FIG. 11G, FIG. 11H, FIG. 11I, FIG. 11J, FIG. 11K, FIG. 11L, FIG. 11M, and FIG. 11N provide schematic representation of nanoparticles and peptides.

FIGS. 12A and 12B provide graphs demonstrating how KGYY₆ may improve glucose tolerance and insulin sensitivity.

FIGS. 13A, 13B, and 13C provide charts demonstrating that 15-mer peptide decreases fructosamine levels in TID dogs.

FIG. 14A and FIG. 14B provide charts showing that 15-mer peptide decreases blood glucose levels in TID dogs.

FIGS. 15A-15F provide several graphs demonstrating the 15-mer peptide may increase c-peptide in TID dogs.

FIG. 16 provides a graph of NOD mice that were hyperglycemic with scrambled peptide, MR1, and KGYY15.

FIG. 17 provides several pictures of mice comparing treated versus untreated mice of the EAE mouse model.

FIG. 18 provides a graph of the size distribution by intensity of an exemplar PLGA particle provided herein.

FIG. 19 provides a graph of the release of PLGA particles over time.

FIG. 20A and FIG. 20B each provide a schematic diagram of the production of a peptide-PLGA nanoparticle and a graph of size distribution.

FIG. 21A and FIG. 21B provide a schematic diagram of the production of a peptide-PLGA nanoparticle and a graph of size distribution.

### DETAILED DESCRIPTION

In certain aspects, the present developments provide compositions of matter, including bioactive peptides and PLGA nanoparticles and heavy metal nanoparticles, that may be useful in treating subjects with a variety of autoimmune disorders, syndromes, or diseases. The conditions may include, but are not limited to, type 1 diabetes, multiple sclerosis, rheumatoid arthritis, systemic lupus erythematosus, chronic obstructive pulmonary disease, atherosclerosis and/or acute respiratory distress syndrome, and further may include Hashimoto's thyroiditis and Graves' disease, pernicious anemia, Addison's disease, chronic active hepatitis, and myasthenia gravis. Moreover, in some scenarios, the bioactive peptides by themselves, or in combination with any of the NPs described here, may be used to provide symptom relief and/or treat issues of the dermis and epidermis such including but not limited to autoimmune related scleroderma, alopecia, keratitis, psoriasis, and other such conditions. The compositions of matter and methods of use thereof that are described and provided herein, may in one aspect be used to modulate or modify the levels or interactions of a CD40 complex and CD154, the ligand for CD40, which may thus provide some relief to a patient or subject that may be afflicted with a syndrome implicated by CD40 and/or CD40 overproduction.

In certain aspects, the developments provided herein also provide methods of making the peptide PLGA nanoparticles and peptide metal nanoparticle conjugates which may be used to treat the diseases, syndromes, or conditions mentioned throughout this disclosure.

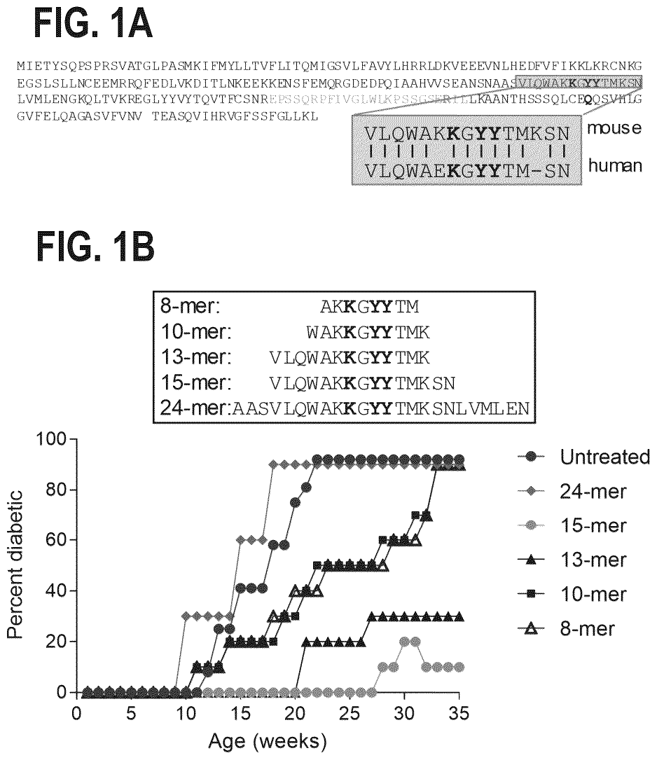

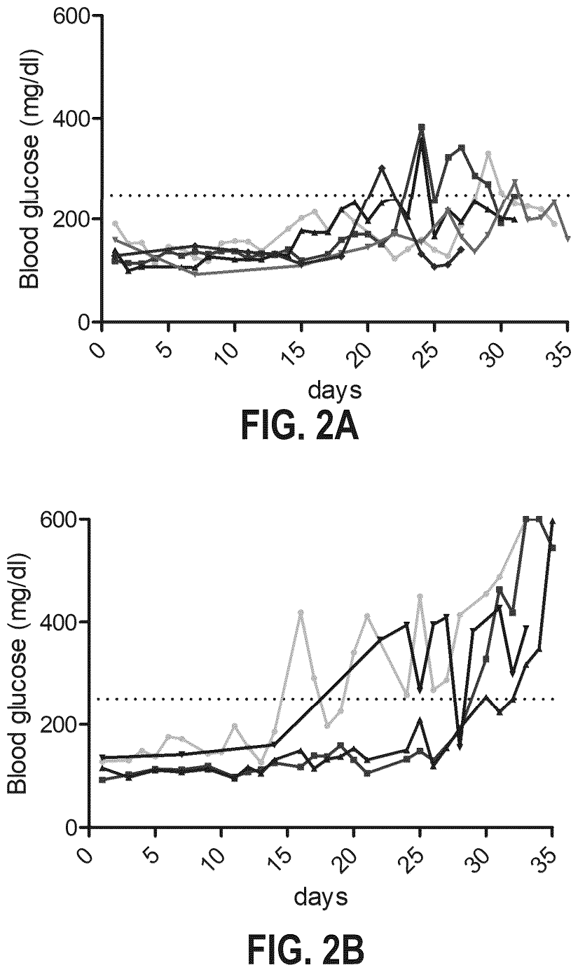

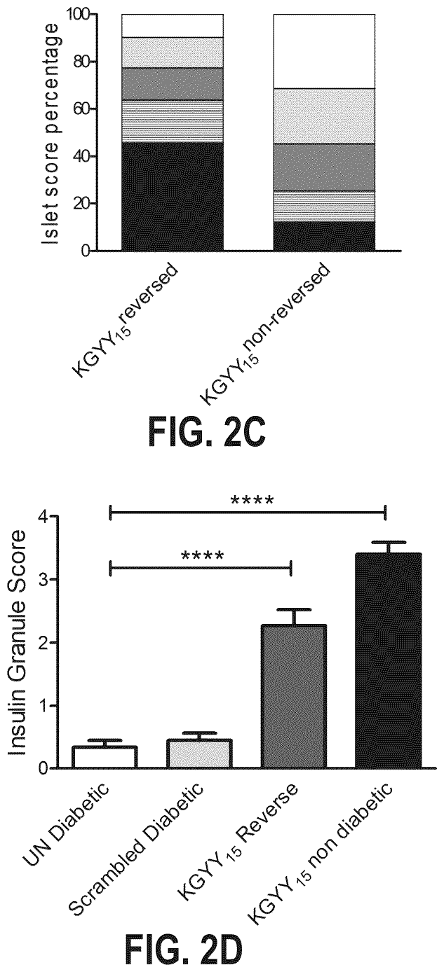

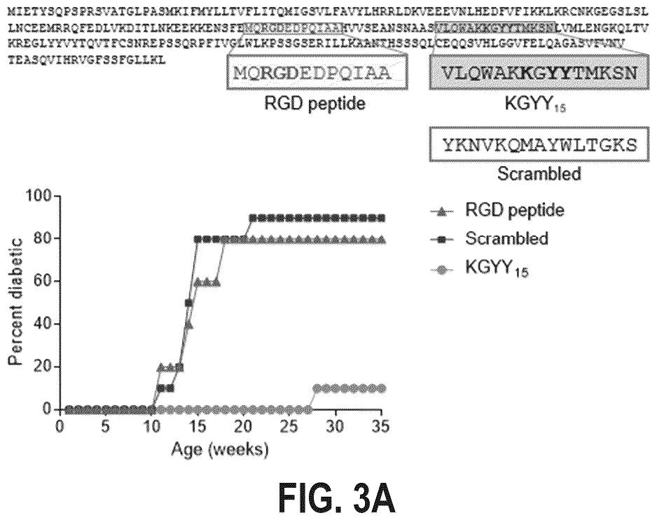

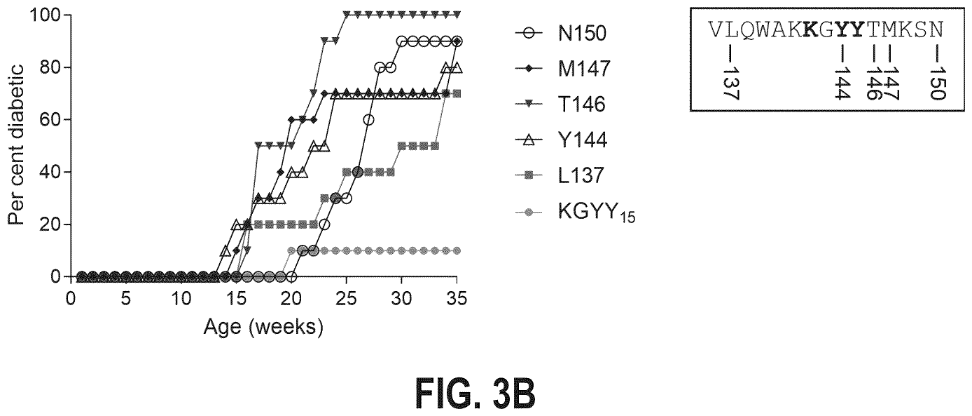

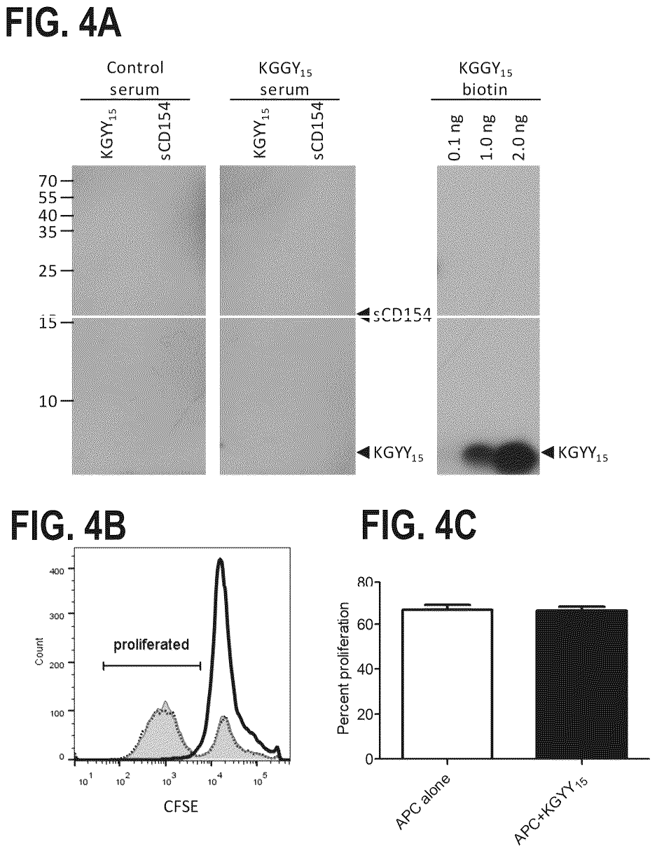

In certain embodiments, approaches disclosed herein utilize peptides that interact with the CD40 protein and the CD40 complex. These peptides may bind to the CD40 complex and thus prevent CD154 from interacting with CD40, and thus alter or block the intercellular signaling and/or intracellular signaling. For example, a 15-mer peptide (KGYY₁₅-VLQWAKKGYYTMKSN) has been utilized to prevent the onset of TID (see FIGS. 1A and 1B) and in one example has shown its ability to even reverse hyperglycemia in approximately 60% of new onset diabetic mice (see FIG. 2). Further, research has demonstrated that five amino acids may be important to these peptides, as shown in FIG. 3. Moreover, KGYY₁₅ does not elicit antibody production in the subject, and thus the KGYY₁₅ peptide does not appear to be immunogenic (see FIG. 4).

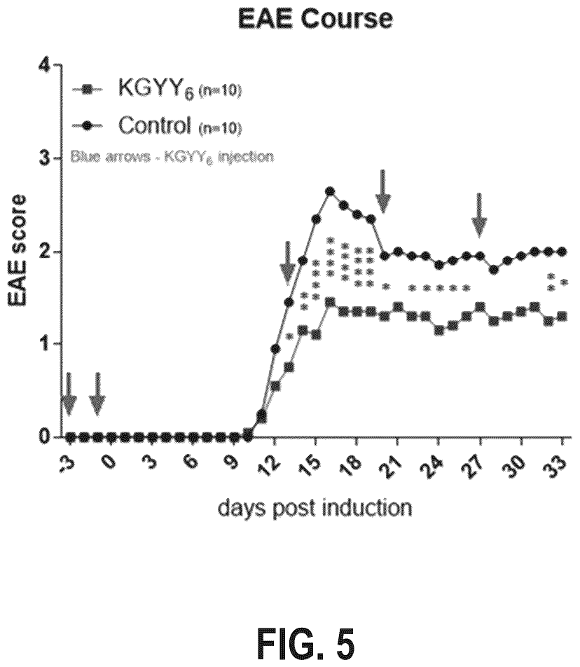

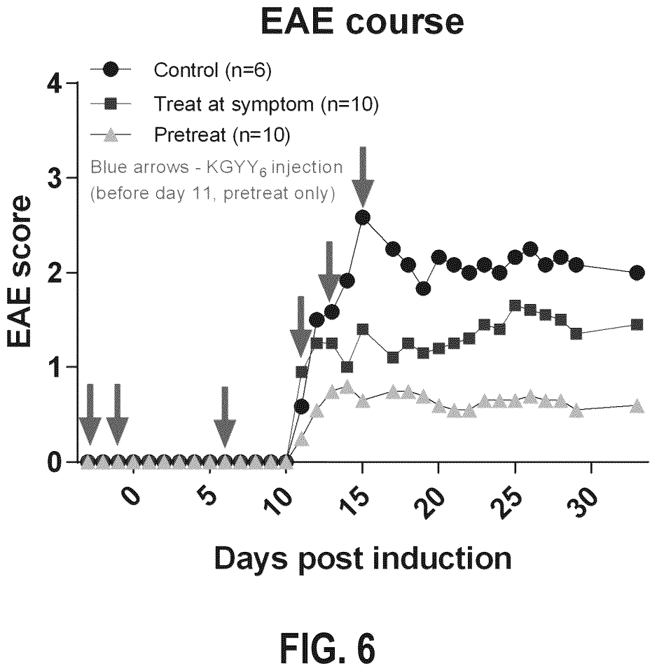

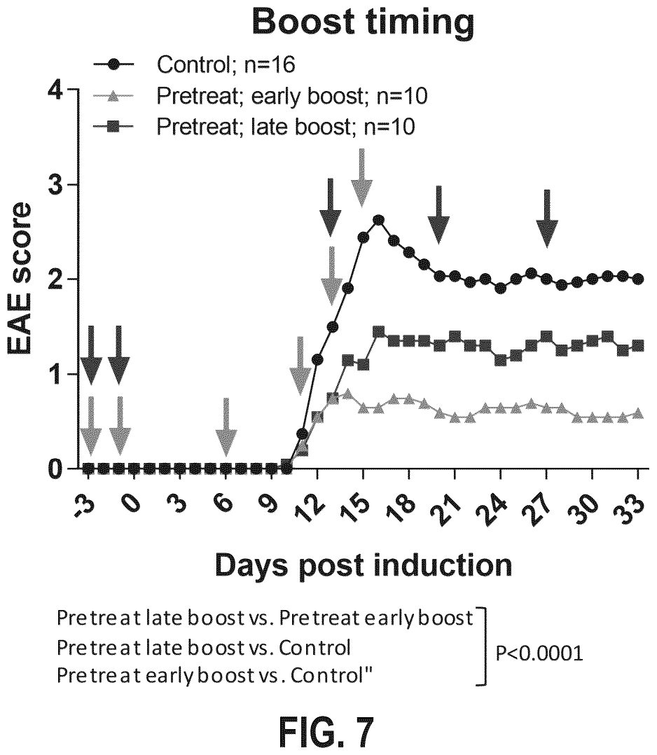

Additionally, a 6-mer peptide (KGYY₆-AKKGYY) has shown efficacy in ameliorating disease in the mouse model of MS. Mouse subjects were pretreated with KGYY₆ peptide prior to disease induction and KGYY₆ boosts were administered when disease symptoms appeared. These studies showed that administration of the 6-mer peptide significantly reduced the severity of the EAE symptoms experienced by the subjects (see FIG. 5). Furthermore, even if no peptide pretreatment is provided and/or peptide treatment initiation is delayed until visible disease symptoms occur, the 6-mer peptide may significantly reduce the severity of EAE (see FIG. 6). Lastly, the earlier the administration of peptide boosts in the EAE disease course, the better the peptide may perform in ameliorating symptoms, especially when compared with simply providing boosters after the onset of visible symptoms (see FIG. 7).

The therapeutic peptides disclosed herein, such as KGYY₁₅ and KGYY₆, have been administered intravenously (i.v.), which may not be the most convenient or ideal, as i.v. administration may necessitate visits to a doctor, clinic; or additional equipment, training, and/or expertise may be needed if administered in an at-home setting. Therefore, alternative routes of administration such as subcutaneous (SC, SQ, sub-cu, sub-Q, SubQ, or subcut) or oral administration may be desirable. One benefit of both subcut and oral administration is that these routes may be performed by the patients themselves without a clinic visit and usually with less equipment, medical training or expertise.

In some embodiments, to implement subcut or oral routes of administration of the therapeutic peptides, peptide drug products, and pharmaceutical compositions, the chemical composition and pharmaceutical formulation may be reformulated. A reformulation may be desirable, helpful, or beneficial because oral administration and subcut administration both have their own unique challenges and hurdles that change the considerations related to the delivery of the therapeutic and bioactive peptide to the target site. For example, for oral route administration, the peptides would pass from mouth to stomach, where stomach acid may dissolve, alter and/or disrupt the structure of the peptide. Further, once passing from stomach to small intestines, the molecular size of the drug product, peptide, conjugates, and pharmaceutical composition may need to be considered and examined as the peptide's size and/or shape may affect whether the peptide is able to traverse the wall of the intestine, either through diffusion or active transport. For subcutaneous administration, less frequent administration may be more desirable; indeed, administration daily or several times a day may not be preferred or ideal. Thus, if a larger dose that is time released can be formulated, a patient may only need to inject subcut peptide on a much less frequent schedule, and in some implementations as a depot. For these and other reasons, nanoparticles (NPs) may be an attractive compositional addition, coupling, or conjugation, that may protect and/or change the release efficiency and dynamics of a drug encapsulated by, conjugated to, or otherwise associated with the NPs.

In certain aspects, the present developments address the need for alternative compositions and methods for treatment and prevention of diseases implicated and affected by the CD40-CD154 inflammatory dyad by describing novel compositions and methods for treatment of diseases for which the compositions provided herein provide therapeutic relief, including but not limited to, type 1 diabetes, multiple sclerosis, rheumatoid arthritis, systemic lupus erythematosus, chronic obstructive pulmonary disease, atherosclerosis and/or acute respiratory distress syndrome. The developments provided herein disclose and describe the use of nanoparticles (NPs) to create nano capsules, nanoparticle drug conjugates, and/or several implementations that utilize these chemical structures for beneficial characteristics and traits.

Before the present developments are further described, it is to be understood that these developments are not strictly limited to particular implementations described, as such may of course vary. It is also to be understood that the terminology used herein is for the purpose of describing particular implementations only, and is not intended to be limiting, since the scope of the present developments will be limited only by the claims.

It must be noted that as used herein and in the appended claims, the singular forms “a,” “an,” and “the” include plural referents unless the context clearly dictates otherwise. It should further be understood that as used herein, the term “a” entity or “an” entity refers to one or more of that entity. For example, a nucleic acid molecule refers to one or more nucleic acid molecules. As such, the terms “a”, “an”, “one or more” and “at least one” can be used interchangeably. Similarly, the terms “comprising”, “including” and “having” can be used interchangeably.

Unless defined otherwise, all technical and scientific terms used herein have the same meaning as commonly understood by one of ordinary skill in the art to which these developments belong. Although any methods and materials similar or equivalent to those described herein can also be used in the practice or testing of the present developments, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present developments are not entitled to antedate such publication by virtue of prior invention. Further, the dates of publication provided may be different from the actual publication dates, which may need to be independently confirmed.

It is appreciated that certain features of the developments, which are, for clarity, described in the context of separate implementations, may also be provided in combination in a single implementation. Conversely, various features of the developments, which are, for brevity, described in the context of a single embodiment, may also be provided separately or in any suitable sub-combination. All combinations of the implementations are specifically embraced by the present developments and are disclosed herein just as if each and every combination was individually and explicitly disclosed. In addition, all sub-combinations are also specifically embraced by the present developments and are disclosed herein just as if each and every such sub-combination was individually and explicitly disclosed herein.

It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

Furthermore, as used herein the term “animal” refers to a vertebrate, preferably a mammal, more preferably a human. Suitable mammals on which to use the methods of the present developments include but are not limited farm animals, sports animals, pets, primates, mice, rats, horses, dogs, cats, and humans. The term “animal” can be used interchangeably with the terms subject or patient.

As used herein, the term “treatment” means a procedure which ameliorates or prevents one or more causes, symptoms, or unwanted/untoward effects a disease or disorder, such as type 1 diabetes, multiple sclerosis, rheumatoid arthritis, systemic lupus erythematosus, chronic obstructive pulmonary disease, atherosclerosis, acute respiratory distress syndrome, Hashimoto's thyroiditis, Graves' disease, pernicious anemia, Addison's disease, chronic active hepatitis, and myasthenia gravis in a subject or patient. Similarly, the term “treat” is used to indicate performing a treatment. Treatments described herein, can, but need not, cure the subject, i.e. remove the cause(s), or remove entirely the symptom(s) and/or unwanted/untoward effect(s) of type 1 diabetes, multiple sclerosis, rheumatoid arthritis, systemic lupus erythematosus, chronic obstructive pulmonary disease, atherosclerosis, acute respiratory distress syndrome, Hashimoto's thyroiditis, Graves' disease, pernicious anemia, Addison's disease, chronic active hepatitis, and myasthenia gravis on the abnormal condition of the subject.

As used herein, the terms “patient” and “subject” and similar phrases can be used interchangeably and are intended to refer to subjects who are at risk for and/or have been diagnosed with one or more disease or disorder, including type 1 diabetes, multiple sclerosis, rheumatoid arthritis, systemic lupus erythematosus, chronic obstructive pulmonary disease, atherosclerosis, and acute respiratory distress syndrome. The terms “healthy subject,” “non-TID subject,” “non-MS subject,” “subject who does not have TID,” “patient who does not have MS”, and similar phrases, are intended to refer to a subject who has not been diagnosed with TID, MS, or another autoimmune condition. A healthy subject has no other acute systemic disease.

As used herein, the term “sample” or “biological sample” includes a sample of any cell type or from any tissue or body fluid, body fluids including, but not limited to: cerebrospinal fluid (CSF), serum, plasma, blood, or fluid from any suitable tissue. In a preferred embodiment, the biological sample is blood or any component of blood (e.g., serum, plasma, etc.).

As used herein the terms “administer,” “administering,” and “administration” are intended to mean introducing at least one compound into a subject. When administration is for the purpose of treatment, the compound or substance may be provided before, during, and/or after the onset of or progression of a symptom or sign of a disease or disorder, including one or more of type 1 diabetes, multiple sclerosis, rheumatoid arthritis, systemic lupus erythematosus, chronic obstructive pulmonary disease, atherosclerosis, and acute respiratory distress syndrome.

As used herein, a “symptom” may be a subjective manifestation of a disease or disorder from the point of view of the patent.

A “sign” on the other hand, is an objective, clinical manifestation of a disease or disorder that is often measurable or quantifiable, e.g. blood glucose level, CD40 levels, Th40 levels, C-peptide level, low blood oxygen levels or rapid respiratory rate. The therapeutic administration of compounds and peptides provided herein may serve to attenuate any symptom or sign or prevent additional symptoms or signs from arising. In at least some instances the substance or compound may be provided in advance of any visible or detectable symptom or sign of type 1 diabetes, multiple sclerosis, rheumatoid arthritis, systemic lupus erythematosus, chronic obstructive pulmonary disease, atherosclerosis, and acute respiratory distress syndrome. The prophylactic administration of the compounds and peptides provided herein may serve to attenuate subsequently arising symptoms or signs or prevent symptoms or signs from arising altogether. For example, a specific peptide provided herein, may be administered subcutaneously to individuals who are “at-risk” because of predisposing conditions and/or who have developing or established type 1 diabetes, multiple sclerosis, rheumatoid arthritis, systemic lupus erythematosus, or atherosclerosis. Alternatively, other implementations of the developments contemplate oral administration that may be used in a wide variety of settings, ranging from administration in an at home setting prophylactically to i.v., subcut, or oral administration in an acute clinical setting for when the circumstances, symptoms, or signs, indicate that a patient may benefit from receiving an administration of the peptide, peptide PLGA nanoparticles, or peptide metal nanoparticles.

The route of administration of the compounds and peptides provided herein includes, but is not limited to, topical, transdermal, intranasal, intralung (insufflation or aerosolization), transmucosal, oral, subcutaneous, intravenous, intraarterial, intramuscular, intraosseous, intraperitoneal, epidural, and intrathecal administration.

In some embodiments, the peptides, compounds, and methods provided herein may be useful in decreasing the severity of a disease or disorder, such as one or more of type 1 diabetes, multiple sclerosis, rheumatoid arthritis, systemic lupus erythematosus, chronic obstructive pulmonary disease, atherosclerosis, and acute respiratory distress syndrome directly and/or by decreasing factors such as autoimmune inflammation, CD40+ T cell mediated inflammation, and/or hyperglycemia.

Furthermore, the peptides provided herein may in some implementations be used to provide relief, alleviate, and/or treat conditions, syndromes, diseases, symptoms, or issues related to a number of diseases of or relating to the epidermis and dermis such as scleroderma, alopecia, keratitis, psoriasis, and other such conditions. In at least some of these conditions, syndromes, diseases, the peptides may be configured or enabled to treat autoimmune related or induced disease.

Thus, in some embodiments, the treatment and/or prevention methods provided herein include administering a therapeutically effective amount of peptides to subjects in need thereof. Peptides, peptide PLGA nanoparticles, and peptides covalently linked to a metal nanoparticles (nanoparticle peptide conjugates) useful in these methods may include peptides that have a least a portion of the amino acid sequence of a CD154 protein, or variant thereof, such that the peptide interacts with CD40 protein or CD40 complex in such a manner as to modulate, affect, interfere, block, change, or otherwise alter the interaction of a CD40 complex with CD154. These peptides may comprise 4, 5, 6, 7, 8, 9, 10, 11, 12, 13, 14, 15, 16, 17, 18, 19, 20, 21, 22, 23, 24, or 25, or more contiguous amino acids from the human CD40 ligand (CD154), and interact with CD40 in such a manner as to modulate, affect, interfere, block, change, or otherwise alter the interaction. The peptides may in certain implementations may preferably include a core sequence of lysine-glycine-tyrosine-tyrosine (KGYY; SEQ ID NO: 3-see Table 1). These peptides may alternatively include at least one sequence selected from the group of SEQ ID NO: 4, 5, 6, 7, 8; SEQ ID NO: 27, 28, 29, 30; and SEQ ID NO: 32, 33, 34. These peptide sequences are set forth in the following Table 1:

TABLE 1

| SEQ ID | | | |
|---|---|---|---|
| NO | SEQUENCE | Description | Length |
| 1 | MIETYSQPSP RSVATGLPAS MKIFMYLLTV | SwissPro 27548.2 | |
| | FLITQMIGSV LFAVYLHRRL DKVEEEVNLH | Mouse CD40 Ligand | |
| | EDFVFIKKLK RCNKGEGSLS LLNCEEMRRQ | (CD154 Protein) | |
| | FEDLVKDITL NKEEKKENSF EMQRGDEDPQ | | |
| | IAAHVVSEAN SNAASVLQWA KKGYYTMKSN | | |
| | LVMLENGKQL TVKREGLYYV YTQVTFCSNR | | |
| | EPSSQRPFIV GLWLKPSSGS ERILLKAANT | | |
| | HSSSQLCEQQ SVHLGGVFEL QAGASVFVNV | | |
| | TEASQVIHRV GFSSFGLLKL | | |
| 2 | MIETYNQTSP RSAATGLPIS MKIFMYLLTV | SwissPro 29965 | |
| | FLITQMIGSA LFAVYLHRRL DKIEDERNLH | Human CD40 Ligand | |
| | EDFVFMKTIQ RCNTGERSLS LLNCEEIKSQ | (CD154 Protein) | |
| | FEGFVKDIML NKEETKKENS FEMQKGDQNP | | |
| | QIAAHVISEA SSKTTSVLQW AEKGYYTMSN | | |
| | NLVTLENGKQ LTVKRQGLYY IYAQVTFCSN | | |
| | REASSQAPFI ASLCLKSPGR FERILLRAAN | | |
| | THSSAKPCGQ QSIHLGGVFE LQPGASVFVN | | |
| | VTDPSQVSHG TGFTSFGLLK L | | |
| 3 | KGYY | Core-sequence | 4 |
| 4 | AKKGYY | 6-mer | |
| 5 | AKKGYYTM | 8-mer-mouse | |
| 6 | AEKGYYTM | 8-mer human | |
| 7 | VLQWAKKGYYTMKSN | 15-mer-mouse | |
| 8 | VLQWAEKGYYTMSNN | 15-mer human | |
| 9 | NAASVLQWAKKGYYTMKSNLVMLE | 24-mer mouse | |
| 10 | ISQAVHAAHAEINEAGR | 15-mer from | |
| | | ovalbumin; control | |
| | | peptide | |
| 11 | G-L-Q-W-A-K-K-G-Y-Y-T-M-K-S-N | Gly-1 | |
| 12 | V-G-Q-W-A-K-K-G-Y-Y-T-M-K-S-N | Gly-2 | |
| 13 | V-L-G-W-A-K-K-G-Y-Y-T-M-K-S-N | Gly-3 | |
| 14 | V-L-Q-G-A-K-K-G-Y-Y-T-M-K-S-N | Gly-4 | |
| 15 | V-L-Q-W-G-K-K-G-Y-Y-T-M-K-S-N | Gly-5 | |
| 16 | V-L-Q-W-A-G-K-G-Y-Y-T-M-K-S-N | Gly-6 | |
| 17 | V-L-Q-W-A-K-G-G-Y-Y-T-M-K-S-N | Gly-7 | |
| 18 | V-L-Q-W-A-K-K-G-G-Y-T-M-K-S-N | Gly-9 | |
| 19 | V-L-Q-W-A-K-K-G-Y-G-T-M-K-S-N | Gly-10 | |
| 20 | V-L-Q-W-A-K-K-G-Y-Y-G-M-K-S-N | Gly-11 | |
| 21 | V-L-Q-W-A-K-K-G-Y-Y-T-G-K-S-N | Gly-12 | |
| 22 | ISQAVHAAHAEINEAGR | 15-mer from | |
| | | ovalbumin; control | |
| | | peptide | |
| 23 | YVQGKANLKSKLMYT | Scrambled peptide | |
| 24 | WAKKGYYTMK | 10-mer mouse | |
| 25 | VLQWAKKGYYTMK | 13-mer mouse | |
| 26 | AASVLQW AKKGYYTMKSNLVMLEN | 24-mer mouse | |
| 27 | KGYYTM | 6-mer (Form 2) | |
| | | human | |
| 28 | AEKGYY | 6-mer (Form 3) | |
| | | human | |
| 29 | KKGYYT | 6-mer (Form 4) | |
| | | mouse | |
| 30 | AKGYYT | 6-mer (Form 5) | |
| | | synthetic | |
| 31 | VKKGYY | 6-mer (Form 6) | |
| | | synthetic | |
| 31 | YKNVKQMAYWLTGKS | Scrambled peptide | |
| 32 | VLRWA PKGYY TISSN | 15-mer - Form 3 | |
| 33 | VLQWA PKGYY TISSN | 15-mer - Form 4 | |
| 34 | VLQWA QKGYY TISNN | 15-mer - Form 5 | |
| 35 | YGRKKRRQRRR | TAT | |
| 36 | XGRKKRRQRRR | TAT variant - | |
| | | where X is an | |
| | | amino acid other | |
| | | than Y | |
| 37 | GRKKRRQRRR | TAT variant | |
| 38 | MIETYSQTAP RSVATGPPVS MKIFMYLLTV | SwissPro O97626 | 260 |
| | FLITQMIGSA LFAVYLHRRL DKIEDERNLY | Canis lupus | |
| | EDFVFMKTLQ KCNKGEGSLS LLNCEEIKSQ | familiaris(Dog) | |
| | FEAFLKEIML NNEMKKEENI AMQKGDQDPR | CD40 Ligand | |
| | IAAHVISEAS SNPASVLRWA PKGYYTISSN | (CD154 Protein) | |
| | LVSLENGKQL AVKRQGLYYV YAQVTFCSNR | | |
| | AASSQAPFVA SLCLHSPSGT ERVLLRAASS | | |
| | RGSSKPCGQQ SIHLGGVFEL HPGASVFVNV | | |
| | TDPSQVSHGT GFTSFGLLKL | | |
| 39 | MIETYSQTAP RSVAPGPPVS MKIFMYLLTV | SwissPro O97605 | 260 |
| | FLITQMIGSA LFAVYLHRRL DKIEDERNLY | Felis catus | |
| | EDFVFMKTLQ KCNKGEGALS LLNCEEIKSR | (Cat) | |
| | FEAFLKEIML NKETKKEKNV AMQKGDQDPR | CD40 Ligand | |
| | VAAHVISEAS SSTASVLQWA PKGYYTISSN | (CD154 Protein) | |
| | LVTLENGKQL AVKRQGLYYI YAQVTFCSNR | | |
| | EASSQAPFIA SLCLHSPSGS ERVLLRAAMA | | |
| | RSSSKPCGQQ SIHLGGVFEL HPGASVFVNV | | |
| | TDPSQVSHGT GFTSFGLLKL | | |
| 40 | MIETYSQPSP RSVATGPPVS MKIFMYLLTV | SwissPro F7AR26-1 | 261 |
| | FLITQMIVSA LFAVYLHRRL DKIEDERNLH | Equus caballus | |
| | EDFVFMKTIQ RCNKGEGPLS LLNCEEIRSQ | (Horse) | |
| | FEGFVKDIML NEEVKKKGEN FEMQKGDQEP | CD40 Ligand | |
| | QIAAHVISEA SSKTASVLQW AQKGYYTISN | (CD154 Protein) | |
| | NLVTLENGKQ LAVKRQGLYY IYAQVTFCSN | | |
| | REASGQAPFI ASLCLRSVSG SERILLRAAN | | |
| | THSSSKPCGQ QSIHLGGVFE LQPGASVFVN | | |
| | VTDPSQVSHG TGFTSFGLLK L | | |
| 41 | KKGYYT | 6-mer - Form 7 | 6 |
| 42 | EKGYYT | 6-mer - Form 8 | 6 |
| 43 | PKGYYT | 6-mer - Form 9 | 6 |
| 44 | QKGYYT | 6-mer - Form 10 | 6 |
| 45 | VLRWA PKGYY TISSN | 15-mer - Form 3 | 15 |
| 46 | VLQWA PKGYY TISSN | 15-mer - Form 6 | 15 |
| 47 | VLQWA QKGYY TISNN | 15-mer - Form 7 | 15 |
| 48 | NAASV LQWAK KGYYT MKSNLVMLE | 24-mer - Form 3 | 24 |
| 49 | APKGYY | 6-mer dog | |

The peptides may have sequences that are entirely responsible for the interaction of the peptide with a CD40 protein or CD40 complex; however, the peptides provided herein may additionally contain amino acid sequences that do not interact with a CD40 protein as they may have other useful functions as well. For example, in addition to the amino acid sequence responsible for interacting with a CD40 protein, a peptide of the present disclosure can contain amino acid sequences that are useful for visualizing or purifying the peptide. Such sequences act as labels (e.g., enzymes) or tags (antibody binding sites). Examples of such labels and tags include, but are not limited to, B-galactosidase, luciferase, glutathione-s-transferase, thioredoxin, HIS-tags, biotin tags, and fluorescent tags. Other useful sequences for labeling and tagging proteins are known to those of skill in the art.

Likewise, in some embodiments, peptides disclosed herein can be modified, so long as such modification does not significantly affect the ability of the peptide to treat type 1 diabetes, multiple sclerosis, rheumatoid arthritis, systemic lupus erythematosus, chronic obstructive pulmonary disease, atherosclerosis, acute respiratory distress syndrome, and/or other autoimmune conditions for which the peptides have shown efficacious results for preventing, reducing, and/or ameliorating symptoms or signs of the disease. Such modifications can be made, for example, to increase the stability, solubility or absorbability of the protein. Examples of such modifications include, but are not limited to pegylation, glycosylation and chemical modification of the peptide. Additional modifications and additions are further described below and relate to using PLGA nanoparticles and metal nanoparticles to modify the drug release, release efficiency, stability, and other characteristics that may be desirable in peptide nanoparticles.

In some embodiments, peptides provided herein may be formed as a fusion with another peptide that may enhance one or more properties of the peptide. In one version, the peptide may be fused to another peptide that imparts favorable pharmacokinetic characteristics to the peptide. In another example, the peptide may be fused with a peptide that enhances the intracellular transport of the therapeutic peptide. Examples of these peptides may include TAT derived from HIV, antennapedia derived from Drosophila, VP22 from herpes simplex virus, complementary-determining regions (CDR) 2 and 3 of anti-DNA antibodies, 70 KDa heat shock protein, and transportan. For example, in at least one implementation the HIV internalization peptide YGRKKRRQRRR (SEQ ID NO: 35) may be utilized. Other known variants of this sequence may also be further implemented to confer some additional properties to be used with the peptide. Additional peptides such as XGRKKRRQRRR (SEQ ID NO: 36), where X is an amino acid other than Y may also be utilized in this manner, thus for example GRKKRRQRRR (SEQ ID NO: 37) is a variant of a peptide that may be used as a TAT variant in implementations provided herein.

Also contemplated in the context of the methods and compositions provided herein is the alteration of the therapeutic peptides by chemical or genetic means. Examples of this type of alteration or modification may include the construction of peptides of partial or complete sequences with non-natural amino acids in L or D enantiomeric forms. Accordingly, any of the peptides provided herein and disclosed herein, and any variants, thereof could be produced in all-D form. Additionally, the peptides provided herein may be further modified to contain carbohydrate or lipid moieties, such as sugars or fatty acids, covalently linked to the side chains of the N- or C-termini of the amino acids. Furthermore, the peptides may be additionally altered or enhanced by glycosylation and/or phosphorylation. Likewise, the peptides provided herein may be acetylated or amidated at N- or C-termini as this may confer desirable properties or characteristics to the peptide.

In addition to the alterations, modifications, and enhancements previously described, the peptides provided herein may be modified to enhance solubility and/or half-life upon being administered. For example, polyethylene glycol (PEG) and related polymers may be used to stabilize the peptide and extend the half-life of the peptide in the blood or patient tissue. A variety of PEG enhancements and modifications are contemplated hereby including two PEG side chains linked via the primary amino groups of a lysine. Moreover, a peptide provided herein may be modified by acetylation on the N-terminus and/or amidation on the C-terminus, which may be used to stabilize the peptide.

In yet another implementation, the peptides provided herein may be linked to one or more domains of an Fc region of mouse, human, canine, feline, or equine, IgG immunoglobin, or the equivalent in each species, and/or may be linked to an epitope tag polypeptide comprising between 6 and 50 amino acid residues. In some instances, the peptides and proteins fused to an Fc region have been observed to exhibit substantially greater half-life in vivo than the unfused counterpart. Additionally, a fusion to an Fc region may allow for dimerization/multimerization of the fusion polypeptide. The Fc region may a naturally occurring Fc region, or may be altered to improve certain qualities, including but not limited to therapeutic qualities, circulation time, and reduced aggregation.

In some implementations, the peptides provided herein may also be modified to contain, conjugated to, and/or administered with phosphorus, sulfur, manganese, magnesium, calcium, halogens, metals, etc. Amino acid mimics may be used to produce polypeptides, and therefore, the polypeptides of this disclosure may include amino acids mimics that have enhanced properties, such as resistance to degradation.

In some embodiments, the peptides and methods provided herein may also comprise administering pro-drugs that metabolize to an active form of these peptides. As used herein, a “pro-drug” is a compound that a biological system metabolizes to an active compound as a result of spontaneous chemical reaction(s), enzyme catalyzed reaction(s), and/or metabolic chemical reaction(s), or a combination of each. Standard prodrugs are formed using groups attached to functionality, e.g. HO—, HS—, HOOC—, R2N—, associated with the drug, that cleave in vivo. Standard prodrugs include, but are not limited to, carboxylate esters where the group is alkyl, aryl, aralkyl, acyloxyalkyl, alkoxycarbonyloxyalkyl as well as esters of hydroxyl, thiol and amines, where the group attached is an acyl group, an alkoxycarbonyl, aminocarbonyl, phosphate or sulfate. The groups illustrated are exemplary and not exhaustive, and one skilled in the art could prepare other known varieties of prodrugs. Prodrugs must undergo some form of a chemical transformation to produce the compound that is biologically active or is a precursor of the biologically active compound. In some cases, the prodrug is biologically active, usually less than the drug itself, and serves to improve drug efficacy or safety through improved bioavailability, pharmacodynamic half-life, etc.

In certain embodiments, the methods of treatment provided herein may include co-administering the peptides with a least one additional compound. The term “co-administer” indicates that each of at least two compounds is administered during a time frame wherein the respective periods of biological activity or effects overlap, but they need not be administered simultaneously. Thus, the term “co-administer” includes sequential as well as co-extensive administration of the compounds of the present developments. Similar to “administering,” “co-administering” of more than one substance may be for therapeutic and/or prophylactic purposes. If more than one substance is co-administered, the routes of administration of the two or more substances need not be the same. In one specific implementation, the peptide is co-administered with a derivative of L-ergothioneine, N-acetylcysteine (NAC), an organoselenium, a thiol-yielding compound, a glutathione enhancing compound and/or alpha-lipoic acid. The development contemplates the possibility of administering a variety of agents alone and/or in combination with the peptide in any order and by any means. The scope of the developments are not limited by the identity of the substance which may be co-administered.

In yet another implementation of treatment provided herein, the peptides may be provided as a sterile solution in 10 mM acetate buffer, in 5% glucose, in water. In this implementation the pH may be approximately 5.5. The peptide in at least one implementation will be provided as a sterile solution in 10 mM acetate buffer, in 5% glucose in water, pH 5.5, 5 mL at 20 mg/mL, 100 mg/vial. On the day of administration, the product will be diluted in saline to result in 50 mL for intravenous (i.v.) infusion over 30 minutes. The developments provided herein further contemplate using alternative buffer (for example phosphate buffered saline (PBS)) systems that may facilitate efficacious delivery and treatment with the peptide.

In some implementations, the peptides provided herein, may be suspended in a pH controlling buffer including phosphate buffered saline (PBS), tris buffered saline, etc. The pH may be adjusted and tuned to a desirable range. Thus the pH may be, for example, 5.5, 6, 6.5, 6, 7.2, 7.5, or any pH that may allow the peptide to perform is desired function. Moreover, in some non-limiting implementations the pH may be adjusted to be in any range prior to encapsulation. Further, in some implementations a salt form of the peptide may be desirable. Thus the peptide may be constructed or produced as an acetate salt, trifluoric salt, hydrochlorate salt, etc.

In some embodiments, one property of the inhibitor peptides provided herein may be that they decrease hyperglycemia development. Thus the methods provided herein may include treating or preventing hyperglycemia in a subject by administering to the subject a composition comprising a peptide of any one of SEQ ID NOs: 4, 5, 6, 7, 8; SEQ ID NO: 27, 28, 29, 30; and SEQ ID NO: 32, 33, 34, and functional fragments and/or analogs thereof, in an amount and under conditions effective to prevent hypoglycemia. Thus, these methods may include the treatment of both non-diabetic and diabetic subjects.

In certain embodiments, the compositions of matter and methods provided herein may also be desirable and suitable to the treatment of a subject developing ARDS or suspected of developing ARDS and/or at risk of developing ARDS. This may also include treatment in order to decrease or prevent hyperglycemia associated with developing ARDS that may exacerbate or intensify the severity or rate of developing ARDS. In these methods, the blood glucose levels of the subject may be reduced to less than 140 mg/dl, or less than 120 mg/dl, or less than 110 mg/dl. In these methods the blood glucose levels of the subject may be maintained greater than 80 mg/dl, or greater than 90 mg/dl, or greater than 100 mg/dl. It should be recognized that these methods are not limited only to treating ARDS but may also be useful in reducing hyperglycemia in the treatment of toxemia, inflammation, infection, bacteremia, sepsis, septic shock, acute lung injury, severe acute respiratory syndrome (SARS), COVID-19, long-COVID-19, systemic inflammatory response syndrome (SIRS), cytokine release syndrome (CRS), type 2 diabetes, or multiple organ dysfunction syndrome (MODS).

Furthermore, ARDS may, in some instances, include what is sometimes described as “cytokine storm”, “cytokine release syndrome”, or “hypercytokinemia”. The peptides provided herein, and the peptides within or associated with NPs may be used to treat this condition by targeting either T cell cytokine producers or antigen producing cells, innate immune cells, producers of macrophages and dendritic cells. The peptides and NP formulations provided herein may also be used to treat endothelium, that produce chemokines to attract immune cells, or produce cytokines to promote localized or systemic inflammation. In some embodiments, the methods and compositions provided herein can be used to treat and/or prevent cytokine release syndrome, for example, in subject suffering from COVID-19 and/or long-COVID-19. “Long-COVID-19” refers to symptoms that last for weeks, months, or longer after initial infection with the SARS-COV-2 virus. These symptoms can include, but are not limited to, fatigue, shortness of breath or difficulty breathing, cough, joint pain, chest pain, memory, concentration or sleep problems, muscle pain or headache, fast or pounding heartbeat, loss of smell or taste, depression or anxiety, fever, dizziness while standing, and worsened symptoms after physical or mental activities

In certain embodiments, when administered or co-administered, the compounds of the present developments are given in a therapeutically effective amount to the subject. As used herein, the phrase “therapeutically effective amount” means an amount that has any beneficial effect in treating and/or preventing the disease, syndrome, condition, or the clinically presenting abnormal or undesired state. Determining the therapeutically effective amount of the active compound of the peptides of the present development may depend on such factors, including but not limited to, the extend to the condition, syndrome or injury to be treated, the age and condition of the subject to be treated, the weight of the subject, the mass of the subject, the approximate surface area of the subject, and other factors that may be considered by a prescribing physician. The term “therapeutic index” or “therapeutic window” refers to the ratio of the dose of a drug or pro-drug that produces a therapeutically beneficial response relative to the dose that produces an undesired response, such as death, an elevation of markers that are indicative of toxicity, and/or pharmacological side effects.

In some embodiments, the peptides used in the methods provided herein may be administered to the subject in a pharmaceutically acceptable carrier, adjuvant, or vehicle. Select examples of pharmaceutically acceptable carriers, adjuvants, and vehicles, which are well-known in the art disclosed in Remington: The Science and Practice of Pharmacy, 21^{st} Ed., Hendrickson, R., et al., Eds., Lippincott Williams & Wilkins, Baltimore, Md. (2006). The selection of a pharmaceutically acceptable carrier, adjuvant, or vehicle will depend on a variety of factors including but not limited to, the route of administration, dosage levels, the age, weight and/or condition of the subject, etc. Additional carriers including PLGA nanoparticles and metal nanoparticles are described in further detail herein.

In some embodiments, the pharmaceutical compositions may be adapted for administration by any appropriate route, for example by oral (including buccal and sublingual), nasal, rectal, intratracheal, aerosol, topical, transdermal, parenteral, subcutaneous, intramuscular, intravenous, or intradermal routes. Such compositions may be prepared by any method known in the art of pharmacy, for example, by admixing the active ingredient with carrier(s) or excipient(s) under sterile conditions. Such compositions may also include liposomal compositions as drug carriers.

In implementations where the pharmaceutical compositions provided herein are adapted for nasal, intratracheal, or aerosolized delivery and administration, the carrier may be a solid and may include a coarse powder having a particle size, for example, in the nanometer range up to the micron range. Additionally, particles may range from about 20 to 500 microns and may be administered via rapid inhalation through the nasal passage from a container of the powder that is held close up to the nose. Suitable compositions wherein the carrier is a liquid for administration as nasal spray or nasal drops may include aqueous or oil solutions of the active ingredient.

In yet another implementation, pharmaceutical compositions may be adapted for inhalation via fine dusts or mists that may be generated by means of various type of metered dose pressurized aerosols, supercritical fluid aerosolization, nebulizers, or insufflators. In yet another implementation, the pharmaceutical compositions may be adapted for oral administration through a strip that dissolves when placed and held in the mouth.

In some embodiments, the size (length—i.e. number of amino acids) of the peptide may matter for disease prevention capacity. For example, results obtained when peripheral blood mononuclear cells (PBMCs) from MS patients were stained for T cells and antigen presenting cells (APC) together with fluorescenated versions of the 6-mer, 8-mer, or 15-mer peptides demonstrated that certain exemplary 15-mer peptides bound better to T cells than APCs; however, exemplary 6-mer and 8-mer peptides stained both T cells and APCs equally well. In some embodiments, use of different length peptides may be advantageous in treatment of different diseases as each disease may be driven primarily by one cell type or another.

In yet another implementation, pharmaceutical compositions adapted for parenteral administration include aqueous and/or non-aqueous sterile injection solution, which may contain antioxidants, buffers, bacteriostats, and solutes which render the formulation substantially isotonic with the blood of the intended recipient; and aqueous and non-aqueous sterile suspensions which may include suspending agents and thickening agents. Excipients which may be used for injectable solutions include water, alcohol, polyols, glycerin, and vegetable oils, for example. The compositions may be presented in unit-dose or multi-dose containers, for example, in sealed ampoules and vials, and may be stored in freeze-dried (lyophilized) condition requiring only the addition of sterile liquid carrier, for example water for injections, immediately prior to use. Extemporaneous injection solutions and suspensions may be prepared from sterile powders, granules, and tablets. The pharmaceutical compositions may contain preserving agents, solubilizing agents, stabilizing agents, wetting agents, emulsifiers, sweeteners, colorants, odorants, salts (substances of the present disclosure may themselves be provided in the form of a pharmaceutically acceptable sale), buffers, coating agents, or antioxidants. Salts may include acetate salts, hydrochloride salts, trifluoroacetate (TFA) salts, sodium chloride salts, or other salts acceptable and useful for the delivery of the peptide. The pharmaceutical compositions may contain copolymer nanoparticles such as poly(lactic-co-glycolic acid) (PLGA), which is a copolymer of poly lactic acid (PLA) and poly glycolic acid (PGA).

Different forms of PLGA can be generated depending on the ratio of the monomers, lactic acid and glycolic acid that is used. In one implementation, a PLGA 50:50 may be utilized. Here, “50:50” denotes a copolymer that is composed of 50% lactic acid and 50% glycolic acid. In other implementations, a PLGA 90:10, a PLGA 80:20, a PLGA 75:25, a PLGA 70:30, a PLGA 65:35, a PLGA 60:40, a PLGA 55:45, a PLGA 45:55, a PLGA 40:60, a PLGA 35:65, a PLGA 30:70, a PLGA 25:75, a PLGA 20:80, and a PLGA 10:90 may be utilized. One characteristic of a co-polymer that may be important is that it is biodegradable, thus allowing the body to process the co-polymer. If a co-polymer such as PLGA is utilized, the body may use hydrolysis to convert the PLGA in to metabolite monomers, lactic acid and glycolic acid, which are normally found in the human body and may be easily metabolized via the Krebs cycle. PLGA may be utilized as it may provide therapeutic results with only minimal systemic toxicity. The degradation time of PLGA may vary but is often dependent on the copolymer ratio as well as the molecular weight of the polymer. PLGA NPs may provide the added benefit of providing a mechanism of delivering a therapeutic peptide intracellularly, instead of simply delivering the peptide extracellularly. PLGA NPs may enter cells via pinocytosis (invagination of the cell membrane) and clathrin-mediated endocytosis (a vesicle formed and coated by the protein clathrin). Once taken up by the cell, the PLGA NPs may be released into the cytoplasm in a short period of time, i.e. 10 minutes.

In one aspect of the developments provided herein, the PLGA NPs provided herein may be formulated with tunable sizes, ranging from nano- to micrometer. This may be accomplished by using D-α-Tocopheryl polyethylene glycol 1000 succinate (simply TPGS or Vitamin E TPGS) in an emulsion technique with ultrasonication in non-miscible solvents followed by evaporation and ultracentrifugation.

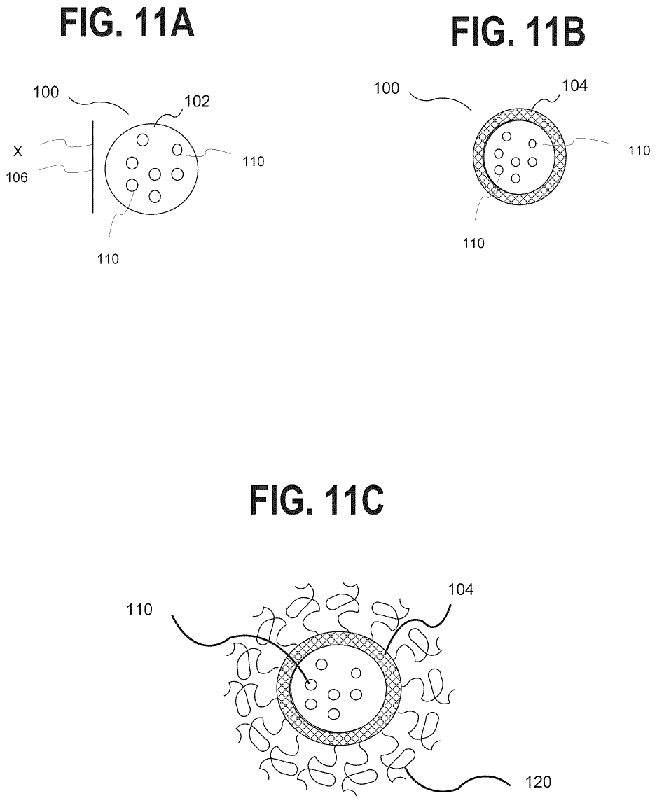

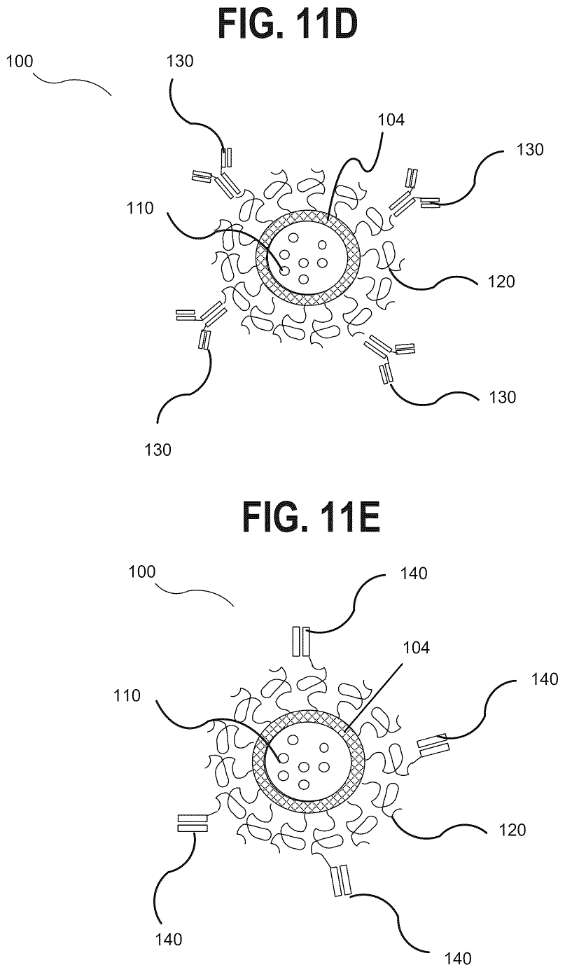

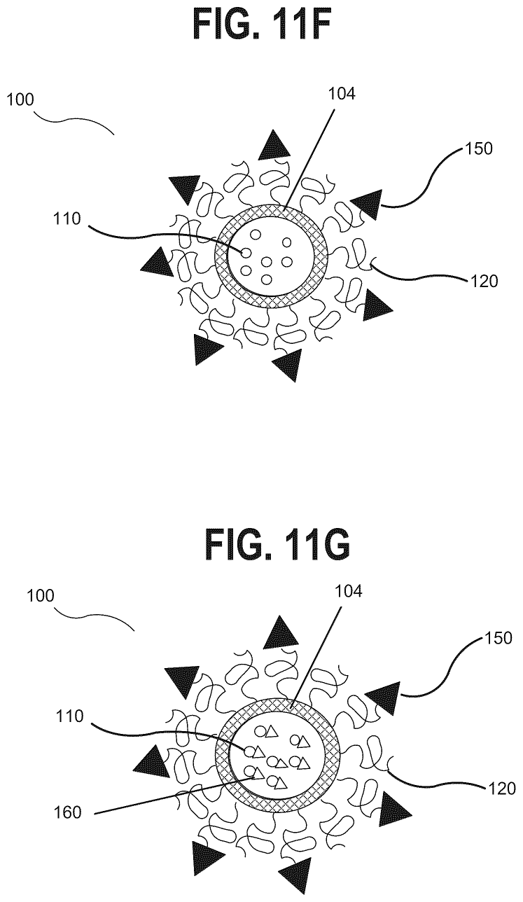

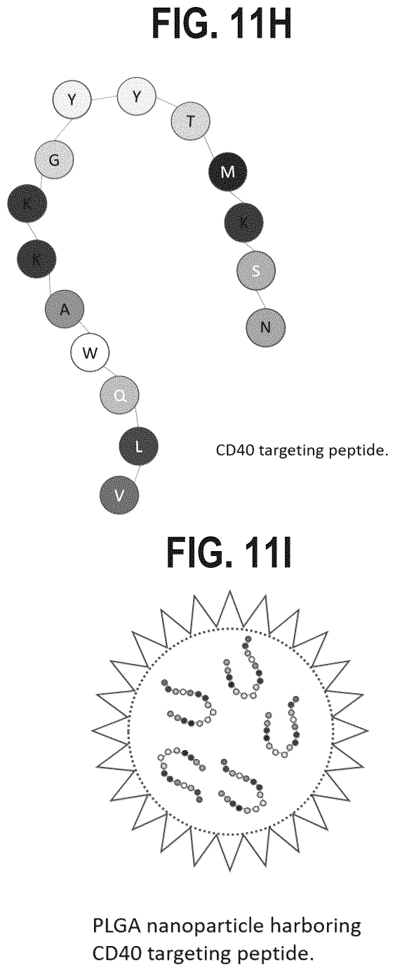

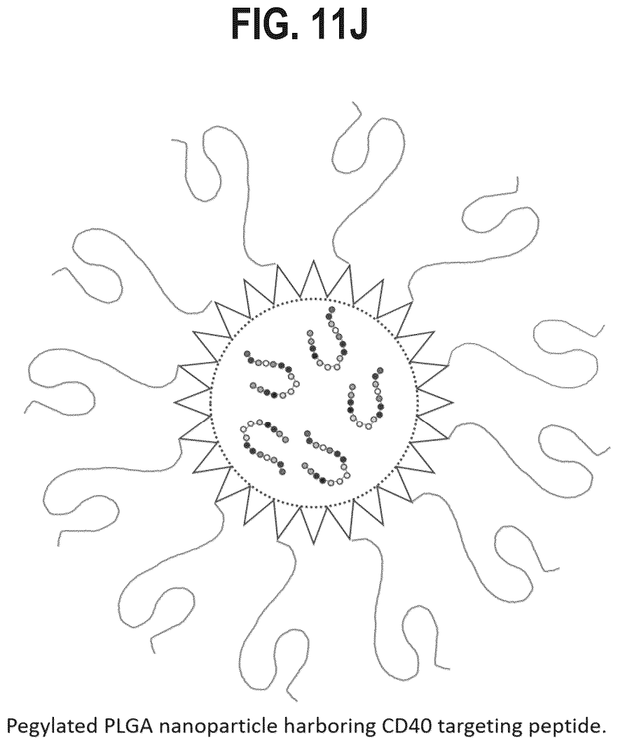

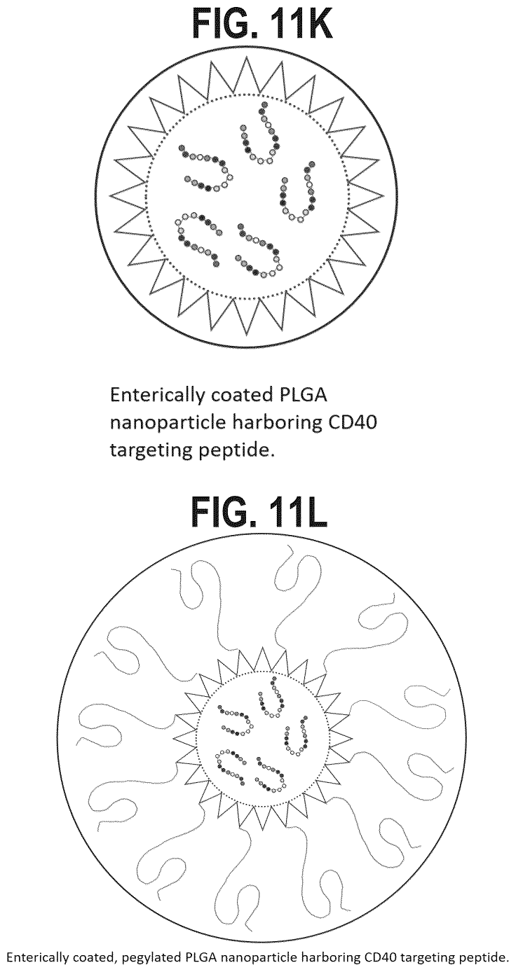

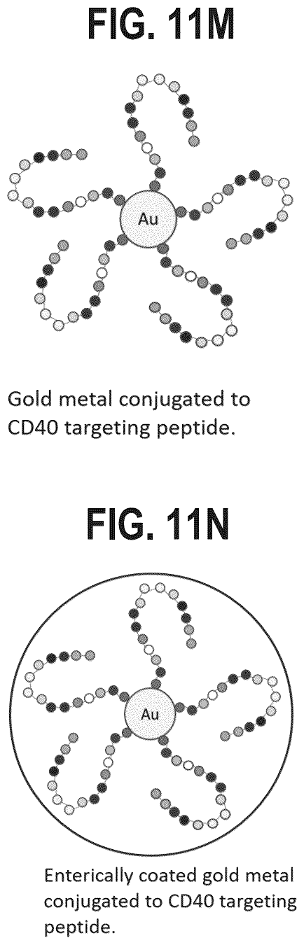

FIG. 11A provides one exemplar of a nanoparticle 100 provided herein. In this example, the nanoparticle is a nanosphere 102 with a dimension or diameter of x, here 106. The dimension or diameter may be 50-200 nm. A drug 110, may be a bioactive peptide, such as those of any of SEQ ID NOs: 4, 5, 6, 7, 8; SEQ ID NO: 27, 28, 29, 30; and SEQ ID NO: 32, 33, 34, and may be encompassed or encased in the nanosphere 102.

FIG. 11B provides another exemplar of a nanoparticle 100 provided herein. In this example, the nanoparticle is a nanocapsule 104. The dimensions of this nanocapsule again, may range from 50-200 nm. A drug 110, may be a bioactive peptide, such as those of any of SEQ ID NOs: 4, 5, 6, 7, 8, 27, 28, 29, 30, 32, 33, and 34. The drug 110 or peptide, may be protected and encapsulated as depicted in the schematic of FIG. 11B.

In some embodiments, different size may be important in distribution and uptake. In some instances, polymer polyethylene glycol (PEG) 120 is used to coat the NPs as in FIG. 11C. The PEG coating of nanoparticles may increase the nanoparticles half-life in blood circulation by several orders of magnitude. PEG exhibits an excellent biocompatibility and “PEGylation” can also be used to give the NPs a positive charge, which may allow a higher extent of internalization as well as escape from lysosomes.

In other instances, the NPs may be coated with PEG molecules 120 and may have IgG 130 attached thereto, such as in FIG. 11D. In this example, a nanocapsule 104 envelops the desired drug 110. The nanocapsule 104 may be coated with polyethylene glycol (PEG) 120. IgG 130 may be further attached or conjugated to the surface the polyethylene glycol molecules. The use of antibodies may allow the nanoparticle, nanocapsule, or nanosphere to be delivered to specific cells or organs.

FIG. 11E provides yet another example of how NPs provided herein may be coated with PEG 120 and has Fab′ 140 regions of an antibody. In this exemplar nanoparticle 100, a nanocapsule 104 contains drug 110, which in implementations provided herein may be one or more variants of a bioactive peptide such as any of SEQ ID NOs: 4, 5, 6, 7, 8, 27, 28, 29, 30, 32, 33, and 34. The use of Fab′ 140 regions of an antibody coupled to a PEG molecule may allow the peptide to be delivered to specific cells or organs.

FIG. 11F provides yet another example of how a nanoparticle 100 provided herein may be further modified to provide additional desirable features. Here, a nanocapsule 104 contains a drug 110, which in implementation provided herein may be one or more variants of a bioactive peptides such as any of SEQ ID NOs: 4, 5, 6, 7, 8, 27, 28, 29, 30, 32, 33, and 34. A targeting ligand 150 is attached, coupled, or associated with the polyethylene glycol (PEG) 120 that provides a coating to the nanocapsule 104. The attachment of a targeting ligand 150 may allow the nanoparticle 100 to deliver the drug 110 to more specific cells as the targeting ligand 150 may allow the nanoparticle 100 to bind to and activate only certain types of cells.

FIG. 11G provides yet another example of how a nanoparticle 100 provided herein may be further modified to provide additional desirable features. Here, a nanocapsule 104 contains a drug 110, and the drug 110 is further coupled to an imaging agent 160. An imaging agent 160 may allow for detection and imaging of the target site. An example of an imaging agent may include iron oxide or derivatives thereof such as dextran-coated iron oxide (ferumoxide) (Feridex®), dextran-coated iron oxide (ferumoxtran) (Combidex®); other imaging agents may include sulfur colloid (Tecnecoll®) and albumin colloid (Nanocoll). Alternatively, a fluorescing molecule may be coupled to the drug 110 to allow for detection and imaging of the target site.

Nanoparticles provided herein may utilize PLGA, TPGS (d-alpha-tocopheryl polyethylene glycol 1000 succinate), and/or PEG in the composition of the nanoparticles. These particular materials may be suitable and useful in that they each may be approved by the US Food and Drug Administration and/or the European Medicines Agency (EMA) for drug delivery in humans.

Nanoparticles provided herein may be generated to have a diameter 0.5 to 20,000 nm. For example, in some embodiments, the nanopartices are between 60-100 nm, 100-200 nm, 200-2000 nm, 2000-3500 nm, 3500-10,000 nm, and 10,000-20,000 nm. The nanoparticles can be loaded with peptides, either a single peptide or a combination of peptide of different sizes, i.e. 6-mer, 8-mer, 10-mer, and 15-mer. A PEGylation or other coating may be employed and in some instances the use of PEGylation may improve distribution and protection of the materials until the NPs reach the target. In some implementations an enteric coating may be used or employed to further coat the NPs. These enteric coatings may include biocompatible fatty acids, waxes, shellac, plastics, and plant fibers. Other enteric coatings may be known to those of skill in the art. This may be even more important when used in oral delivery due to the number of environments and enzymes that the nanoparticle may encounter between ingestion through the mouth, the stomach, and the small intestines. Once administered, whether orally or subcutaneously, or otherwise, the NPs may begin to hydrolyze, causing the slow release of peptide while the monomers of the PLGA may enter the Kreb's cycle for metabolization. The NPs delivery may be desirable in that a more steady dose may be delivered over time rather than a large bolus dose that then declines rapidly over time, until the next dose.

Nanoparticles provided herein may be formed in several different ways including but not limited to thermal annealing, sol-gel process, cross-linking polymerization, inner-core etching method, and solvent emulsion or solvent displacement. PLGA nanoparticles can be formulated to a tunable size using an emulsion technique followed by ultrasonication in non-miscible solvents followed by evaporation and ultracentrifugation.

One implementation of the present development provides a conjugate comprising one or more nanoparticles covalently linked to a therapeutic peptide; wherein, the nanoparticle is selected from the group consisting of gold, silver, copper, platinum, and mixtures thereof, and a peptide that binds to CD40. In one embodiment of the present development, the nanoparticle is zinc oxide nanoparticles. In one aspect, the conjugate can be used in the delivery of therapeutic peptide to a targeted cell or tissue. In certain embodiments, the nanoparticles are in the range of 0.5-70 nm. In certain embodiments, the nanoparticles are 50 nm. The conjugates provided herein comprise nanoparticles, for example gold nanoparticles (Au NPs), which are preferably 50 nm or less in diameter. In certain embodiments, the nanoparticles are less than 50 nm in diameter. The nanoparticles may be less than 50 nm, less than 40 nm, less than 30 nm, less than 20 nm, less than 10 nm, less than 5 nm, less than 2 nm, and less than 1 nm in diameter. In some embodiments, the metal nanoparticle may be as small as, or smaller than the therapeutic peptide. In certain embodiments, the nanoparticles being relatively small in size may be important to how the peptides function or ability to bind CD40 presenting cells. In one embodiment of the present development, the nanoparticle is conjugated to the peptide through thiolation. The use of nanoparticles such as gold nanoparticles to form a conjugate may improve the therapeutic efficacy of the peptide, and in particular the KGYY peptide motif. One aspect of using functionalized gold particles is that the immune response, toxicity, and in vivo clearance may be well tolerated by subjects. Another aspect of the present development is a method for modulating TID tolerance mechanisms comprising, forming a conjugate comprising one or more nanoparticles covalently linked to a therapeutic peptide that binds CD40, and administering the conjugate to a patient in need of a therapeutic amount thereof.

The NPs provided herein may be characterized using scanning electron microscopy (SEM), transmission electron microscopy (TEM), dynamic light scattering (DLS), confocal microscopy analysis, and UV-visible spectrophotometry to determine the particle size distribution. Fourier-transform infrared (FTIR) spectroscopy may be used to confirm the peptide functionalization of the Au NPs. The loading of peptides on Au NPs may also be characterized using thermogravimetric and UV-Vis with fluorescence coupled high-performance liquid chromatography (HPLC). In one embodiment of the present development, the conjugate may be administered to the subject via an injection and observed in vivo using systems such as micro computed tomography (micro-CT) or other imaging technology. Nanoparticle size may also be analyzed using dynamic light scattering.

One implementation of the present development provides a hollow poly(lactic-co-glycolic acid) (PLGA) nanoparticle containing, enveloping, or harboring a peptide that binds to CD40. In certain embodiments, the hollow PLGA nanoparticles are in the range of 60-100 nm, 100-200 nm, 200-2000 nm, 2000-3500 nm, 3500-10,000 nm, and 10,000-20,000 nm. In certain embodiments, the PLGA nanoparticles are 80 nm. The PLGA nanoparticles may be less than 100 nm, less than 90 nm, less than 80 nm, less than 70 nm, less than 60 nm, less than 50 nm, less than 40 nm, less than 30 nm, less than 20 nm, less than 20 nm, less than 10 nm, or less than 5 nm in diameter.

PLGA NPs may be produced by a method by adding a known concentration of peptides (e.g. 6-mer, 15-mer, or other peptide that binds CD40) to an ethyl acetate-PLGA solution. These PLGA NP-peptide conjugates may then be characterized using TEM, DLS, FTIR, and HPLC to determine the size, distribution, and peptide loading.

One aspect of the present development is a method for modulating TID tolerance mechanisms comprising, forming a PLGA NP conjugate comprising one or more PLGA nanoparticles covalently linked to a therapeutic peptide that binds CD40, and administering the PLGA NP conjugate to a patient in need of a therapeutic amount thereof. Markers of autoimmune diseases, such as type 1 diabetes, multiple sclerosis, rheumatoid arthritis, systemic lupus erythematosus, chronic obstructive pulmonary disease, atherosclerosis, acute respiratory distress syndrome, may be known in the art in some instances. For example, in TID, markers some researchers postulate that markers may include but are not limited to pro-insulin autoantibody (IAA), glutamic acid decarboxylase 65 (GAD), insulinoma antigen-2 (IA-2), and zinc transporter 8 (ZnT8) autoantibodies. (See World Wide Web at c-path.org/programs/t1d/tools-and-projects/t1d-biomarkers/). These autoantibodies, or their presence in a subjects blood, may, in combination with blood glucose measurements, aid in the identification and classification of TID subjects. In other autoimmune diseases such as MS, clinicians and practitioners routinely utilize MRIs to assess disease and disease progression; however, emerging research has begun to identify a number of molecular biomarkers that may be measured through one or more assays for measuring each of the markers that may useful in assessing the baseline and disease progression.

In the instance that ARDS and alveolar capillary membrane injury may be the condition that afflicts the individual subject, other markers known in the art may be assessed. These markers include but are not limited to the presence of inflammatory cytokines. Examples, of inflammatory cytokines may include, but are not limited to, interleukin-1 (IL-1), interferon gamma (IFNγ), tumor necrosis factor alpha (TNFα), and interleukin-8 (IL-8). Other cytokines such as IL-6, IL-17A, and GM-CSF may also provide relevant markers related to such a syndrome. Assaying for specific cytokines may be well known in the art and may include assays such as ELISA assays, multiplex assays, Northern blots, Western blots, activity assays, and the like. Other signs and markers of ARDS and alveolar capillary membrane injury may include, but are not limited to, changes in blood pressure, increased number of lung neutrophils, increased levels of lung GSSG, increased concentration of expired H2O2, increased levels of lactate dehydrogenase (LDH) and a lung leak index. Other markers can be assayed using morphological assays of lung injury, physiologic derangements including measurements of hypoxia, and abnormalities of including GSH depletion, alterations of GSH/GSSG ration, increased measurements of oxidative stress, e.g. increased levels of 8-iso-PGFα. IL-6 is also recognized as being associated and indicative of ARDS. Assays for measuring each of the markers of ARDS, and alveolar capillary membrane injury may be known in the art.

One embodiment of the present development provides a conjugate comprising one or more nanoparticles covalently linked to a therapeutic peptide; wherein, the nanoparticle is selected from the group consisting of gold, silver, copper, platinum, and mixtures thereof, and a peptide that binds to CD40. In one embodiment of the present development, the nanoparticle is zinc oxide nanoparticles. In one aspect, the conjugate can be used in the delivery of therapeutic peptide to a targeted cell or tissue. In certain embodiments, the nanoparticles are in the range of 0.5-70 nm. In certain embodiments, the nanoparticles are 50 nm. The conjugates provided herein comprise nanoparticles, for example gold nanoparticles (Au NPs), which are preferably 50 nm or less in diameter. In certain embodiments, the nanoparticles are less than 50 nm in diameter. The nanoparticles may be less than 50 nm, less than 40 nm, less than 30 nm, less than 20 nm, less than 10 nm, less than 5 nm, less than 2 nm, and less than 1 nm in diameter. In some embodiments, the metal nanoparticle may be as small as, or smaller than the therapeutic peptide. In certain embodiments, the nanoparticles being small in size may be important to how the peptide agents function or ability to bind CD40 presenting cells. In one embodiment of the present development, the nanoparticle is conjugated to the peptide through thiolation. The use of nanoparticles such as gold nanoparticles to form a conjugate may improve the therapeutic efficacy of the peptide, and in particular the KGYY peptide motif. One aspect of using functionalized gold particles is that the immune response, toxicity, and in vivo clearance may be well tolerated by subjects. Another aspect of the present development is a method for modulating TID tolerance mechanisms comprising, forming a conjugate comprising one or more nanoparticles covalently linked to a therapeutic peptide that binds CD40, and administering the conjugate to a patient in need of a therapeutic amount thereof.

The NPs used may be characterized using transmission electron microscopy (TEM), dynamic light scattering (DLS), and UV-visible spectrophotometry to determine the particle size distribution. Fourier-transform infrared (FTIR) spectroscopy may be used to confirm the peptide functionalization of the Au NPs. The loading of peptides on Au NPs may also be characterized using thermogravimetric and UV-Vis with fluorescence coupled high-performance liquid chromatography (HPLC). In one embodiment of the present development, the conjugate may be administered to the subject via an injection and observed in vivo using systems such as micro-CT or other imaging technology.

One embodiment of the present development provides a hollow poly(lactic-co-glycolic acid) (PLGA) nanoparticle linked to a peptide that binds to CD40. In certain embodiments, the hollow PLGA nanoparticles are in the range of 60-100 nm. In certain embodiments, the PLGA nanoparticles are 80 nm. The PLGA nanoparticles may be less than 100 nm, less than 90 nm, less than 80 nm, less than 70 nm, less than 60 nm, less than 50 nm, less than 40 nm, less than 30 nm, less than 20 nm, less than 20 nm, less than 10 nm, or less than 5 nm in diameter.

PLGA NPs may be produced by a method by adding a known concentration of peptides (e.g. 6-mer, 15-mer, or other peptide that binds CD40) to an ethyl acetate-PLGA solution. These PLGA NP-peptide conjugates may then be characterized using TEM, DLS, FTIR, and HPLC to determine the size, distribution, and peptide loading.

One aspect of the present development is a method for modulating TID tolerance mechanisms comprising, forming a PLGA NP conjugate comprising one or more PLGA nanoparticles covalently linked to a therapeutic peptide that binds CD40, and administering the PLGA NP conjugate to a patient in need of a therapeutic amount thereof.

The following examples are provided for purpose of illustration and are not intended to limit the scope of the present invention.

### EXAMPLES

### Example 1

FIGS. 1A and 1B demonstrate that several lengths of peptide were tested in NOD mice. A 15-mer CD154-derived peptide prevents diabetes. (a) The amino acid sequence of mouse CD154 is depicted, with amino acids known to interact with CD40 shown in bold. A 15 amino acid sequence is highlighted in grey and aligned with the same motif in human CD154. A previously studied peptide is highlighted. (b) NOD mice were treated with different sizes of peptides, as indicated (n=10 in all groups). Mice were monitored for diabetes onset (blood glucose>250 mg/dl). Differences were significant by two-way ANOVA; p<0.0001.

### Example 2

FIG. 2A-2D are based on a study of the use of KGYY₁₅ to treat NOD mice. KGYY₁₅ reverses overt hyperglycemia (a, b) Starting at 10 to 12 weeks of age (day 0), blood glucose was monitored in NOD mice. Immediately after diabetes onset (blood glucose>250 mg/dl; n=9), mice were treated daily with KGYY₁₅ and blood glucose was monitored. FIG. 2A is a graph that depicts each mouse that underwent hyperglycemia reversal; FIG. 2B is a graph depicts those that did not. FIG. 2C is a graph showing Islet insulitis scores in reversed and non-reversed mice: 0, no infiltration (black); 1, 25% infiltration (striped); 2, 50% infiltration (dark grey); 3, 75% infiltration (light grey); 4, 100% infiltration (white). FIG. 2D is a graph showing Islet insulin granulation scores: 0, less than 25% granulation; 1, 25-50% granulation; 2, 50-75% granulation; 4, 75-100% granulated. Data is represented as the mean±SEM; **** p<0.0001 by two-tailed unpaired t test.

### Example 3

FIG. 3A shows an example of a study where an RGD peptide, scrambled peptide, and KGYY 15 peptide were administered to NOD mice over a period of 35 weeks. The percent of subjects that developed diabetes in the KGYY₁₅ subjects was much lower, as compared with the RGD peptide and the scrambled peptide. KGYY₁₅ diabetes prevention is sequence dependent, and several amino acid positions are critical for its function. (a) KGYY₁₅-treated mice were compared with mice treated with a scrambled peptide or a peptide targeting the RGD region (sequences indicated above the graph). (b) KGYY₁₅-treated mice were compared with mice treated with a 15-mer peptide series in which single amino acids, as indicated, were substituted for glycine. L137; Y144; T146; M147; N150; n=10 in all groups shown in (a) and (b), except for L137, where n=9. Differences were significant by two-way ANOVA; p<0.0001

### Example 4

FIGS. 4A-4C demonstrate that KGYY₁₅ does not elicit an apparent immune response to itself or CD154. NOD mice were injected weekly for 7 weeks with 1 mg/kg of KGYY₁₅. In FIG. 4A, serum was collected from injected or control mice and used, followed by anti-mouse-IgG secondary antibody, in western blots for detection of KGYY₁₅ peptide or soluble CD154 (sCD154; KGYY₁₅ and sCD154 sizes are indicated with arrows). An even longer exposure also revealed no bands demonstrating that no antibodies to the peptide had been generated in the treated mice. As a standard, KGYY₁₅-biotin was run and detected with streptavidin-HRP. Experiment was done twice, pooling serum from 2 and 3 mice in each experiment, respectively. In FIGS. 4B and 4C, sorted splenic APC from KGYY₁₅ treated mice were loaded with KGYY₁₅ or not. T cells from KGYY₁₅ treated mice were CFSE labelled then exposed to the autologous APC (+/−KGYY₁₅) and incubated for 5 days. Proliferation was assessed by CFSE dilution and no proliferation was detected. Representative histogram of CFSE dilution: Black line—T cells alone; Grey tinted—T cells+APC alone; Dotted line—T cells+APC loaded with KGYY₁₅. Graph depicts cumulative data (n=5 in each group). The data demonstrate that the peptide is not immunogenic.

### Example 5

One group of mice-C57BL/6 mice from Taconic (Hudson, NY, USA) received KGYY₆ (SEQ ID NO: 4) intravenous (IV) injections 3 days and 1 day prior to EAE induction, while another control group received a vehicle of phosphate buffered saline (PBS). EAE was then induced and the mice started to show symptoms on day 10, which is standard for these mice under these conditions. This is further depicted in FIGS. 11A-11N. On days 13, 20, and 27, the KGYY₆ group of mice receive a boost of peptide, while the other group again received PBS via IV injection. The disease course was significantly less severe in the KGYY₆ treated group compared to the control group (see FIGS. 1A-1B, p<0.0001; two-way ANOVA (Analysis of Variance)). By day 13, there was a statistically significant difference that lasted throughout the experiment. In FIGS. 11A-11N—*-asterisks-denote p-value: *-one asterisk is <0.05; **-two asterisks is <0.01; ***-three asterisks is <0.001; and four asterisks is <0.0001. This is a two-way ANOVA with Sidak's multiple comparisons test. Arrows in above lines in FIGS. 11A-11N represent days −3, −1, 13, 20, and 27, which correspond to days on which the mice received the KGYY₆. Mice were disease scored daily in this study as described in more detail below.

The EAE mouse model can be highly variable even when attempting to control all possible parameters. This variability in EAE may be due to seasonal variations as well as litter differences in the mice. In order to minimize litter-to-litter and seasonal variations, experiments and studies were performed that had 10 mice per group and the mice were randomly mixed once they arrived from the vendor. Female 10-12 week old C57BL/6 mice were immunized subcutaneously on the upper back/neck with 100 μl completely emulsified MOG35-55 peptide (50 μg in 50 μl PBS) and complete Freund's adjuvant (CFA; 75 μg M. Tuberculosis H37 RA (Becton Dickinson and Company (Franklin Lake, NJ, USA)) in 50 μl incomplete Freund's adjuvant (mineral oil)), followed by i.p. pertussis toxin (PT; 200 ng (Sigma-Aldrich® (St. Louis, MO, USA)) in 100 μl PBS) injections. Mice were then randomly assigned to a treatment cohort or vehicle control cohort. One cohort of mice received intravenous injections of the KGYY₆ (SEQ ID NO: 29)-25 μg in 100 μl PBS per mouse on days −3, −1, 13, 20, and 27 as described above. Day 0 is the reference starting point for EAE induction. Another cohort received peptide on days −3, −1, 6, 11, 13, and 15. Vehicle control mice received injections of PBS.

All mice were monitored daily for disease and scored in the following manner: 0—no abnormalities; 0.5—clutching hind limbs; 1—limp tail or weak hind limbs and/or wobbly gait; 1.5—limp tail and clutching hind limbs; 2—limp tail and weak hind limbs and/or wobbly gait wherein the mouse supports itself and propels itself using hind limbs; 2.5—limp tail and weak hind limbs and/or wobbly gait wherein the mouse cannot support and propel itself using hind limbs, but the paws are moving. 3—limp tail and one weak hind limb, while the other hind limb is completely paralyzed wherein the mouse uses the weak hind limb to propel itself in some fashion. 3.5—limp tail and one weak hind limb, while the other hind limb is completely paralyzed where in the mouse does not use the weak hind limb, which is almost at paralysis. 4—limp tail and complete hind limb paralysis wherein the mouse often displays spastic hypertonia and involuntarily crosses its hind limbs. 5—complete paralysis of hind quarter and weak forelimb(s). The data are reported as the mean daily clinical score for all animals in each group. Mice reaching a level 5 or losing more than 20% of their bodyweight are euthanized.

The results of this study are shown in FIG. 6 and FIG. 7 and demonstrate that the administration of KGYY₆ ameliorates EAE, and that earlier boosts of the peptide may have a greater impact.

### Example 6

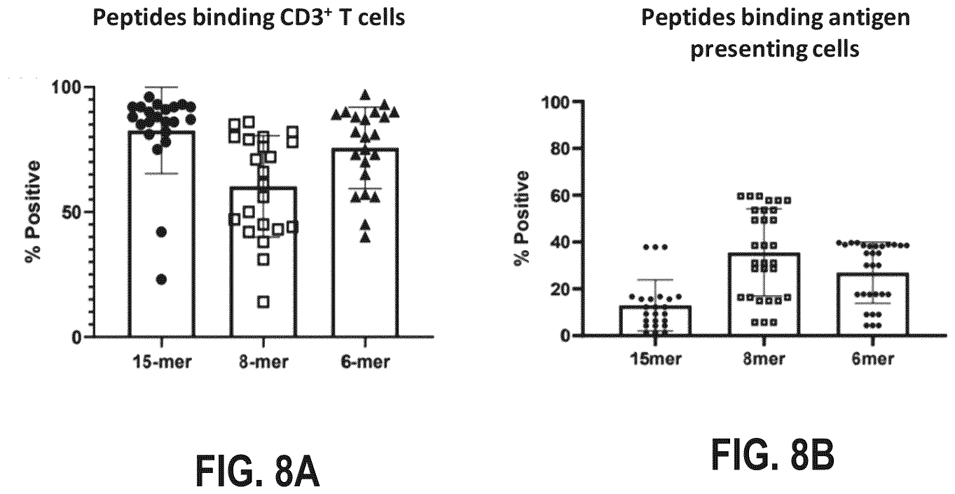

This example shows that fluorescent peptides bind to CD3+ T cells or antigen presenting cells differently depending on the size of the peptide. Peripheral blood mononuclear cells from MS patients were stained then gated on CD3⁺ for T cells or on CD3⁻ for antigen presenting cells (APC) were analyzed for peptide binding levels. Different cells bind different peptides with different intensities as determined by flow cytometry. There were 24 subjects in each test. The results are shown in FIGS. 8A and 8B.

### Example 7

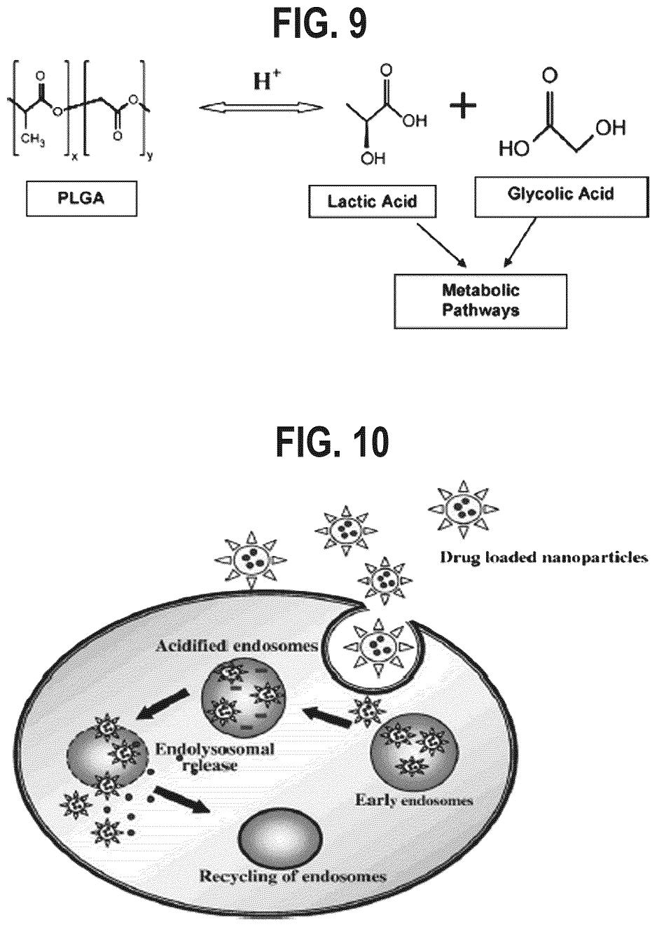

FIG. 9 shows the biodegradation chemical reaction of PLGA into its monomers lactic acid and glycolic acid. The change in PLGA properties during biodegradation influences the release and the degradation rates of incorporated, loaded, or coupled drug molecules. PLGA physical properties may depend on multiple factors, including the initial molecular weight, the ratio of lactide to glycolide, the size of the particles, exposure or non-exposure to water, and storage temperature.

### Example 8

FIG. 10 provides a schematic of nanoparticle internalization in cells. Drug loaded nanoparticles may be delivered to specific tissues or cells and be internalized. In this aspect peptides provided herein may be able to be delivered intracellularly in addition to the surface of cells.

### Example 9

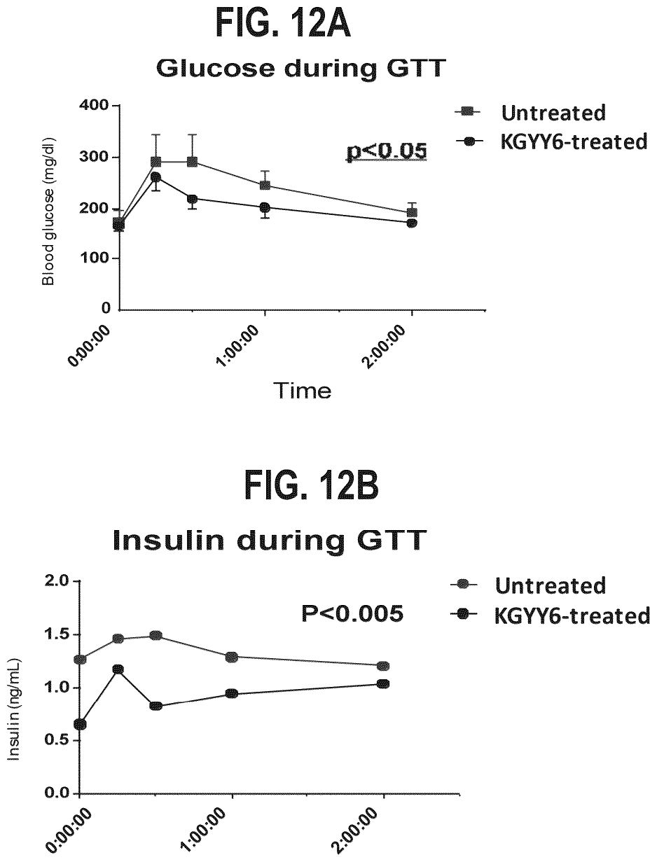

This example demonstrates that KGYY₆ peptide induces a positive change in glucose and insulin in glucose tolerance testing. FIG. 12A and FIG. 12B demonstrate that KGYY₆ improves glucose tolerance and insulin sensitivity. Graphs represent a statistically significant improvement in glucose tolerance (A; p<0.05) and insulin sensitivity (B; p<0.005). ApoE−/− mice (n=14).

### Example 10

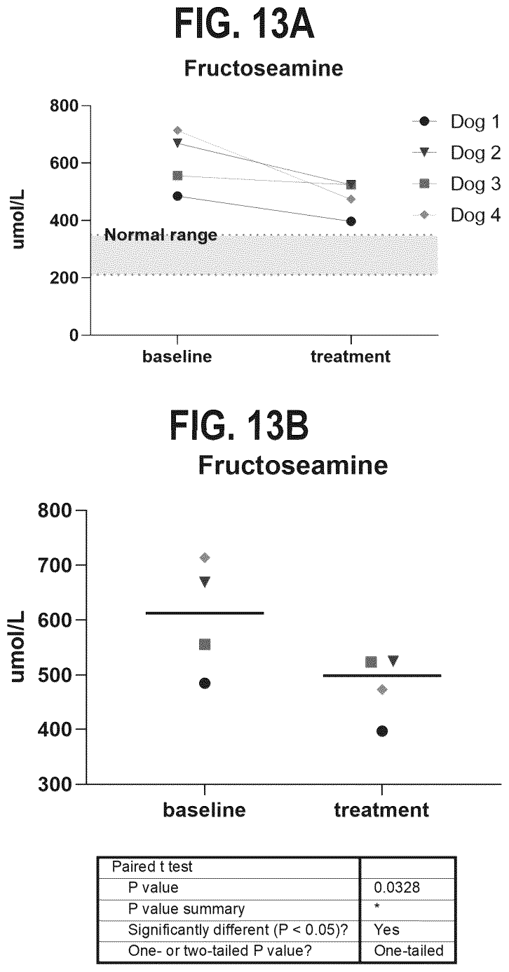

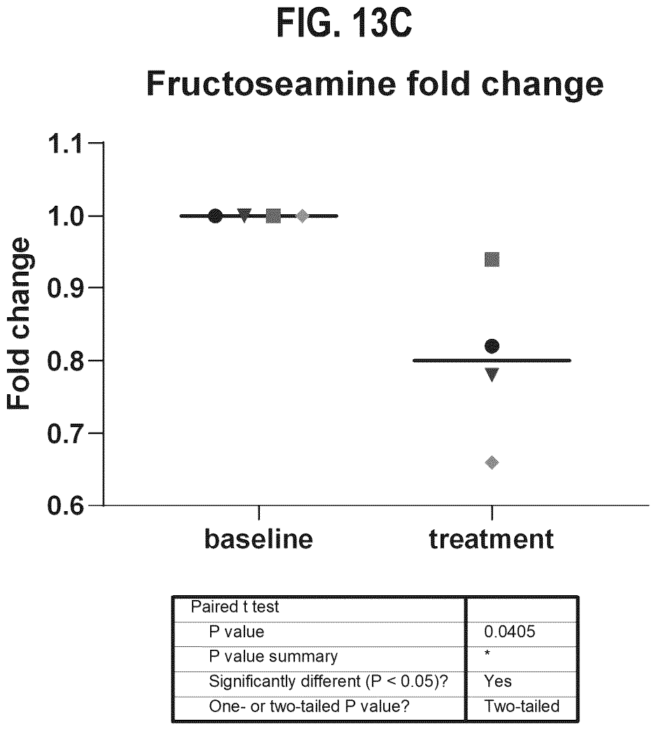

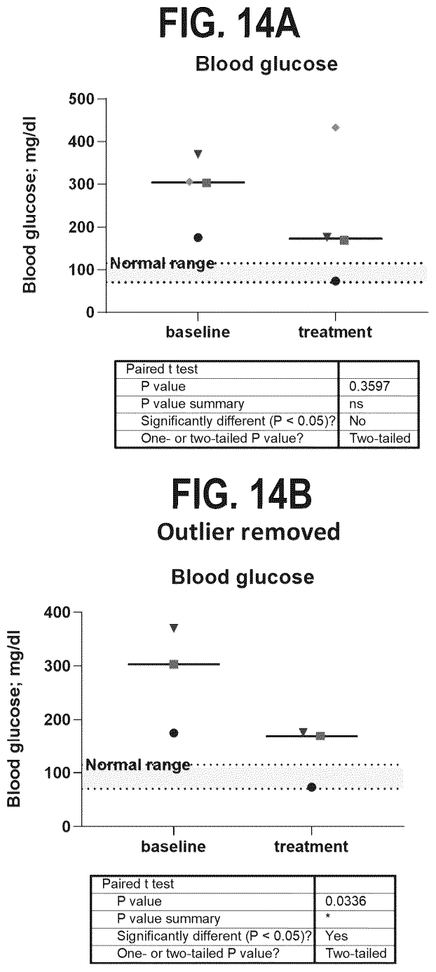

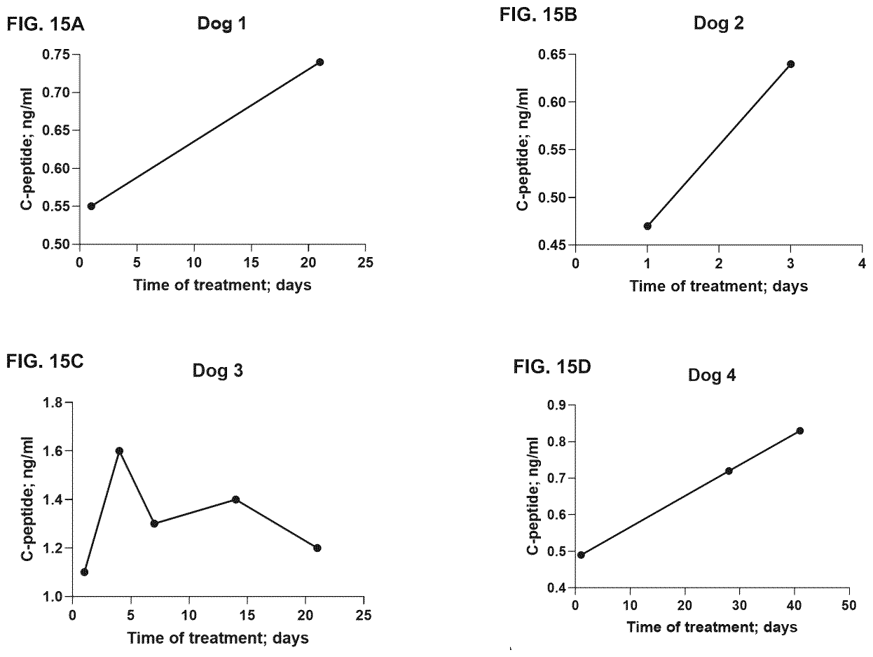

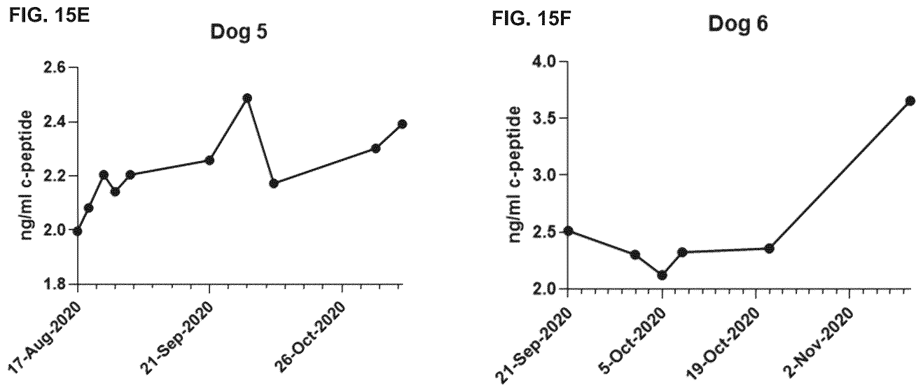

FIGS. 13A, 13B, and 13C demonstrate that Dog 15-mer peptide treatment significantly decreases fructosamine levels in TID dogs. Four longstanding TID domestic dogs were treated with i.v. infusions of dog 15-mer peptide, twice in the first week then weekly thereafter. Levels of fructosamine were measured before the first infusion and several weeks after initiation of treatment. FIG. 14A and FIG. 14B provide charts showing that 15-mer peptide decreases blood glucose levels in TID dogs. FIGS. 15A-15D provide several graphs demonstrating the 15-mer peptide may increase c-peptide in TID dogs. FIGS. 15E-15F provide graphs demonstrating that the 15-mer peptide (SEQ ID NO: 45) and PLGA nanoparticle may increase c-peptide levels in TID dogs.

### Example 11

This example provides a method and description of one way in which PLGA nanoparticles may be synthesized. Here, a solvent displacement technique can be used in preparing the drug loaded PLGA nanoparticles. 10 mg of target peptide and 50 mg of PLGA are be dissolved in 5 ml acetone. The organic phase with a constant flow rate of 0.3 ml/min can be added up into 15 ml of aqueous phase containing 1% of PVA or hydrophilic stabilizer 1.5% of poloxamer 188 under magnetic stirring. With the help of a rotavapor, the organic solvent can be evaporated under vacuum. The suspension can be filtered by using 0.2 μm membrane and centrifuged at 15,000 rpm for 1 hr at 5° C. (Refrigerator centrifuge, Remi C-24). The sediment obtained is again dissolved in distilled water and centrifuged with the same conditions in triplicate. The final product can be dried using a freeze dryer overnight.

Encapsulation efficiency and drug loading can be measured. Nanoparticles (10 mg) of known weights are soaked in 10 ml of phosphate buffer for 30 minutes. The whole solution is centrifuged at 16000 rpm at 15° C. for 30 min to remove the polymeric debris. Polymeric debris is washed twice with fresh solvent to extract any adhered drug in this case target peptide. The clear supernatant solution can be analyzed for drug content by UV spectrophotometer (UV Pharmaspec 1700, SHIMADZU). The complete extraction of drug can be confirmed by repeating the extraction process on the already extracted polymeric debris. The percentage of encapsulation efficiency of the semi-IPN matrix can be calculated. The standard curve of the drug can be obtained by plotting the concentration from 1 μg/ml to 5 μg/ml against its respective absorbance at predetermined light frequency.

The percentage of drug loading and entrapment efficiency was calculated by using the following formula:

Encapsulation efficiency (%)=Amount of drug released from the lyophilized PLGA NPs/Amount of drug initially taken to prepare the NPs×10₀

Drug loading (%)=Amount of drug found in the lyophilized NPs/Amount of lyophilized NPs×100

Particle size analysis can be performed by dynamic light scattering (DLS) using a Malvern Zetasizer 3000 HSA (Malvern Instruments, UK). DLS yields the mean diameter and the polydispersity index (PI) which is a measure of the width of the size distribution. The mean diameter and PI values can be obtained at an angle of 90° in 10 mm diameter cells at 25° C. Prior to the measurements all samples should be diluted with double distilled water to produce a suitable scattering intensity.

By loading NP of different sizes, time-release kinetics will be altered. The same amount of drug will be loaded into each sized NP. By administering a bolus dose of varying sized NPs, a time release schedule can be established. In vivo administration studies followed by pharmacokinetic/pharmacodynamic studies will determine how quickly drug product appears.

NPs may be used to develop a subcut route of administration for bioactive peptide drug administration. NPs also may be used to develop an oral route of administration for drug product.

In addition, a single emulsion/evaporation method can be used to generate particles sized between 200 and 2000 nanometers, based on the concentration of emulsification catalyst used.

The method will generate particles of different sizes, loaded with identical amount of target drug. Loading efficiency will be calculated based on efficiency of target drug absorption at each size particle. The release kinetics of target drug at each sized particle can be determined by ultra-violet-visible spectrometry.

It should be noted that different amounts (by weight, mass, or volume) may be loaded into different amounts (by weight, mass, or volume) of nanoparticles. That is, the above should be considered a non-limiting example as here it is described as 10 mg of peptide and 50 mg of PLGA; however, in other implementations it may be 5 mg of peptide and 50 mg PLGA; 20 mg of peptide in 50 mg of PLGA; 5 mg of peptide in 30 mg PLGA; 20 mg of peptide in 70 mg of PLGA; and these particular ratios may only be limited by the physical limitations of particular ratios of peptide to PLGA which may be calculated, determined, or approximated by one of skill in the art. Thus alternative implementations may be constructed or synthesized by mixing different amounts that may vary the dose of drug and may also have an effect on the time of release and/or release efficiency.

### Example 12

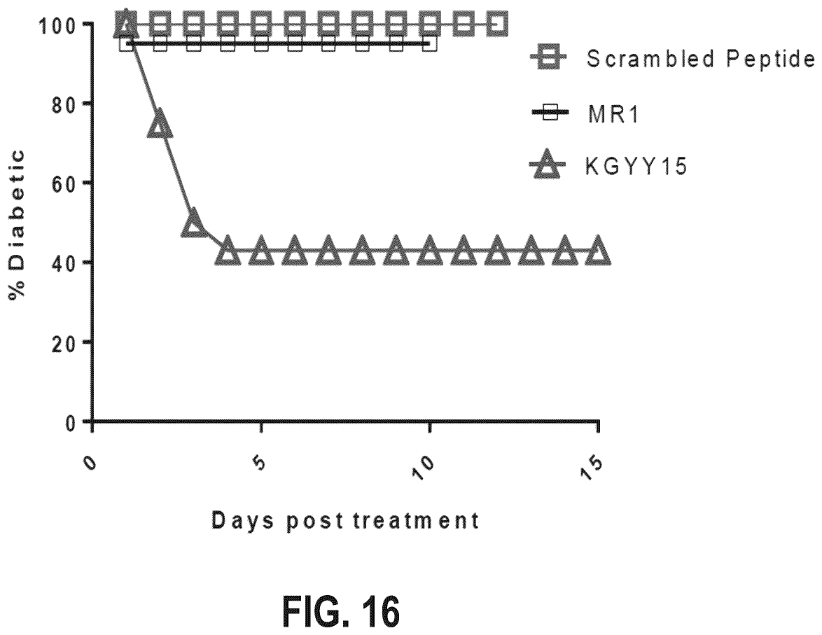

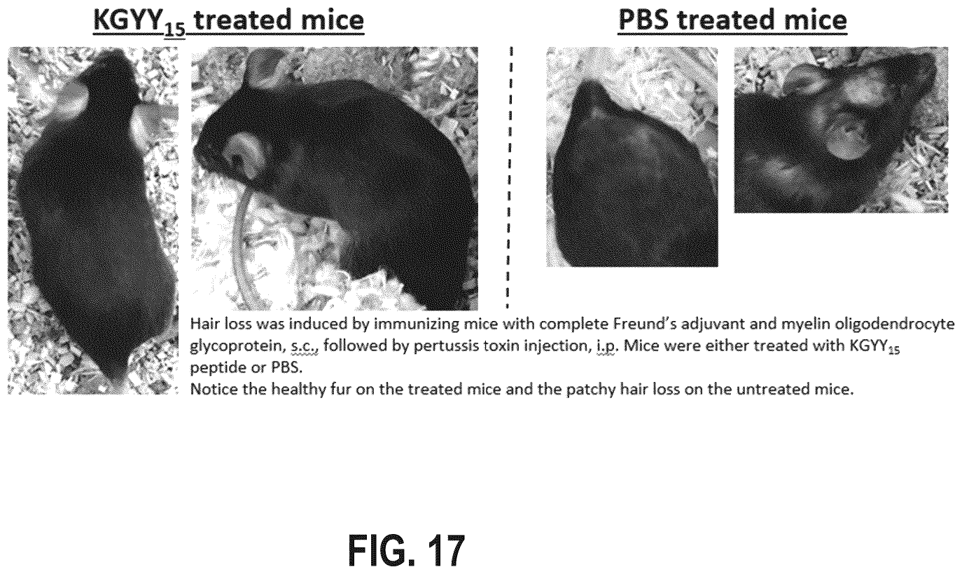

NOD mice that were hyperglycemic, blood glucose level at or greater than 250 mg/dl (and in some cases as high as 450 mg/dl) were administered peptide at a dose of 4 mg/kg daily for 5 days. Controls included a scrambled version and anti-CD154. Monoclonal antibody—MR1—was administered to select subjects of the population because it has been shown to be preventative of diabetes in some instances such as when administered in an early stage, less than 3 weeks of age, but is not effective when administered during mid/late stage pre-diabetes, at approximately 9 weeks of age. MR1 treatment was unable to reverse hyperglycemia; however, administration of 15-mer peptide reduced blood glucose levels to normal (i.e. blood glucose less than 120 mg/dl) for 60% of the treated mice after one week. The results of this study are shown in FIG. 16.

### Example 13

The BDC2.5 T cell clone is highly diabetogenic. The BDC2.5 TCR transgenic mouse created the expectation of rapid and full breach of tolerance leading to TID development. However, BDC2.5.TCR.Tg mice initially proved to be a poor model for breaking tolerance. At 25 weeks of age, when over 80% of a standard NOD colony developed diabetes, only 20% of BDC2.5 untreated mice break tolerance to develop TID. Prior studies have shown that 100% of BDC2.5 mice do break tolerance given sufficient time (45 weeks) and that diabetes onset is rapidly progressed when CD40 engaging protocols are used. One tolerance mechanism involved pathogenic effector cells, defined as CD4+CD40+ or Th40 cells, losing FOXP3 expression in a CD40 mediated manner. These cells were not classic Tregs, but did express FOXP3 being a cell regulator not just a transcription factor associated with Tregs.

NOD mice are treated with CD40 peptides, Au—NP CD40 conjugates and PLGA-NP CD40 conjugates to compare administration techniques. Peptide functionalized NPs (Au NPs and PLGA-NPs) are injected via IV (intravenous), IP (intraperitoneal), or sub-cutaneous (SC) routes, into mice at a concentration of 25 μg equivalent peptide and repeated for 16 weeks. At this point, approximately 80% of control subjects should be diabetic. Bare Au NPs and bare PLGA NPs without any peptides attached, bare peptides without NPs attached, and NPs with scrambled or irrelevant peptides will be used as controls. A positive anti-CD154 will also be used as a control.

At treatment termination, Th40 cells (the cell population that demonstrates pathogenic effector properties) will be isolated. Cells at concentrations of 1, 2.5, and 5×106 are transferred to NOD.scid recipients. Untreated Th40 cells from diabetic BDC2.5 mice transfer hyperglycemia to recipients within 2 weeks and therefore are used as controls. Recipient mice will be monitored weekly for hyperglycemia over 12 weeks. Prior to transfer, a portion of cells will be withheld for TCR examination using RT-PCR to determine the Vα and Vβ TCR constituents. At experimental termination, pancreatic, lymph nodes, and spleen are collected and T cells characterized. TCR analysis is repeated on these cells. Pancreata from all mice subjects are examined for infiltrates. If cells from treated mice do not induce hyperglycemia, the NP-peptide conjugates successfully tolerize pathogenic effector T cells, long term. TCR analysis may determine that if changes in TCR occur including if a specific TCR+ cell expands or contracts in the number in recipients. If cells treated at higher concentration, of either peptide alone, or NP-peptide conjugate, tolerize, but cells at lower concentrations do not, then efficacious treatment doses are determined. Differences in TCR and other characteristics defined below will be determined. If diabetes onset delays, as compared to controls, but eventually occurs, then tolerance induction is temporary.

Tolerized cells will be fully characterized and compared to non-tolerized cells including differences in TCR expression. Differences between bare peptide, Au—NP peptide conjugate, and PLGA-NP peptide conjugate are addressed. This determines whether either of the NP approaches (i.e. AU-NP peptide conjugate or PLGA-NP peptide conjugate) improves tolerance, modulation, or shows signs of improvements or differences of modulation, regulation, or any results that may be different from the bare peptide. Cell surface markers including activation, memory (effector and central), and naïve markers are examined. FOXP3 expression will be analyzed. Rather than being a marker exclusively for regulatory T cells (Tregs), FOXP3 may be viewed as a regulator of cell functions, promoting a non-inflammatory phenotype within the cell independently of Treg status. Cytokine expression and chemokine receptor expression will be determined. Collected cells will be CD3, CD3+CD28, CD3+CD40, and CD3+CD40+CD28 engaged then cytokine panels for Th1, Th17, Th2. Treg associated cytokines will be done by intracellular stains and cytokine assay kits. The measured levels of these cytokines may be important to understanding the tolerance mechanism affects for an array of cytokines. For example, the relationship of Th1 and Th17 relative to Th2/Treg cytokines and each other may elucidate additional mechanisms and modes of action that are not currently appreciated. Aspects of this example may be important to determining additional mechanisms of T cell tolerance, block CD40-mediated auto-inflammation, and duration of tolerance. This example may further show the effects on a defined pathogenic effector T cell subset, Th40 cells, separated completely from Treg influence.

### Example 14

NOD mice are treated with CD40 peptides, Au—NP CD40-peptide conjugates and PLGA-NP CD40 peptide formulations and/or conjugates to compare administration techniques. Peptide functionalized NPs (Au NPs and PLGA-NPs) are injected via IV (intravenous), IP (intraperitoneal), or sub-cutaneous (SC), into mice at a concentration of 25 μg equivalent peptide and repeated for 16 weeks. At this point, approximately 80% of control subjects should be diabetic. Bare Au NPs and bare PLGA NPs without any peptides attached, bare peptides without NPs attached, and NPs with scrambled or irrelevant peptides will be used as controls. A positive anti-CD154 will also be used as a control.

Cells will be removed and isolated from mice at 3, 6, 9, and 12 weeks after treatment commences. Cell isolation will be through blood draws and in some instances some mice are sacrificed to examine pancreas, pancreatic lymph nodes, and spleen. Cells including Th40 and classic Treg cells (i.e. CD4+, CD25 hi, CD127lo, and Helios+, are isolated by cell sorting and levels of Foxp3 are determined. In BDC2.5 TCR transgenic mice, almost all Th40 cells express Foxp3 when mice are young and have not yet broken tolerance, yet those cells are not Tregs. As tolerance is broken, Th40 cells lose Foxp3 expression in a CD40 dependent manner. Accordingly, Foxp3 expression is measured and analyzed via flow cytometry and western blots.

For direct cellular effects, BDC2.5 mice are treated, where Th40 cells express then lose Foxp3 to break tolerance, are treated, as described in Example 13. Th40 and Treg cells are isolated from BDC2.5 and from NOD mice ages 3 weeks (no insulitis), 6 weeks (moderate insulitis), 12 week (extensive insulitis) and after diabetes onset. Cells are analyzed for Foxp3 expression.

Results may show that CD40 targeting peptides may induce Foxp3 as a regulator of effector functions. The treatment of subjects with CD40 targeting peptides, Au—NP CD40 targeting peptide conjugates and PLGA-NP CD40 targeting peptide conjugates, may show results that the nanoparticle conjugate approaches improve Foxp3 levels either as a control mechanism for effector cells or by induction of classic Tregs. Further, this example may show if the CD40 targeting peptide alone induces Foxp3, or if in vivo conditions created by tolerogenic peptide treatment affects Foxp3 expression. Foxp3 will be determine in the Th40 (pathogenic effector) cells and in classic Tregs. In the instance that Foxp3 is induced in the Th40 cells, cells will be cultured for up to 1 week to determine how long Foxp3 is stable. Additionally, classic Tregs may be assayed to determine and compare FOXP3 duration in those cells. Moreover, both cell types may be treated with inflammatory cytokines, including, IFNγ, TNFα, and IL-6. Alternatively, cells will be treated and engaged with CD40 antibodies and analyzed for Foxp3 reduction.

The mechanism of action relative to Foxp3 may be due to the ability of the KGYY-motif peptide to prevent CD40 signals that down-regulate Foxp3. If FOXP3, is induced in any cell type, the subject mice will be injected 1 time with a bolus of the CD40 targeting peptide alone, the AuNP-peptide conjugate, the PLGA-NP-peptide conjugate, and a nanoparticle (either Au or PLGA) alone. At days 2, 4, 8, 12, 24, and 30, cells will be isolated and assayed. This approach may determine how long Foxp3 expression lasts in vivo. For confirmation, NOD.FOXP3YFP/cre mutant mice will be observed and tested for Foxp3 induction long term.

### Example 15

To assess tolerance mediated in mice treated with CD40 targeting peptides, Au—NP CD40 targeting conjugates and PLGA-NP CD40 targeting conjugates, Th40, CD4+CD40- and classic Tregs will be isolated from mice that were treated as described in Example 13. Antigen presenting cells (APCs) from NOD.scid mice will also be isolated because these mice truly have naïve APCs. APCs will be loaded with whole islets, islets that have been disrupted with proteins partially digested, insulin peptides, pro-insulin peptides, insulin c-peptide, GAD67 and GAD65 peptides. T cells will be pre-labelled with tracker dyes like CFSE (Carboxyfluorescein succinimidyl ester—a common fluorescent staining dye) for use in a proliferation assay. Cells that produce IFNγ, for example, that do not produce IFNγ or produce a non-inflammatory cytokine like IL-4 or IL-10 post treatment, would be considered to be tolerized. Therefore, examination and analysis of cytokine production by T cells after exposure to each antigen provides additional support of the modulation that each antigen may provide.

At days 2, 4, 8, 12, 24, and 30, T cells will be isolated and assayed. If cells are not responsive to APC and antigens, then tolerance is likely induced. T cells from untreated, no tolerance induction, will be used as controls. Cells from mice treated for longer time periods may be tolerized while cells from m ice treated for shorter time periods may not. Additional investigation may be needed to determine length of time for the administration of the peptide for tolerance mechanisms to be actuated. This example may show that when CD40 is blocked early by the administration of therapeutic peptides, the TCR repertoire is affected to be more well tolerized. This study may show that block CD40 mediated signals may allow for activation induced cell death (AICD) in some self-antigen response Th40 cells, or it may promote survival of other T cells.

### Example 16

Th40, CD4+CD40- and classic Tregs will be isolated from mice treated with CD40 targeting peptides, Au—NP CD40 targeting conjugates and PLGA-NP CD40 targeting conjugates, described in Example 13. Cells will be subjected to AICD protocols and cell death measured. Fas/Fas-ligand mediated cell death is examined. Cells are exposed to activation conditions including CD3 and CD3/CD28 interactions, with and without Fas engagement.

In addition, APC cells are isolated and fed diabetes associated antigens including islet antigens, insulin, GAD, another known diabetic antigens. AICD is then measured in cells treated with CD40 targeting peptides, Au—NP CD40 targeting conjugates and PLGA-NP CD40 targeting conjugates.

In both instances described in this Example 20, cell death is determined using propidium iodide staining, with and without annexin V stains, and analyzed by flow cytometry.

Additionally, naïve Th40 cells are isolated from NOD mice, exposed to CD40 targeting peptides, Au—NP CD40 targeting conjugates and PLGA-NP CD40 targeting conjugates, and then exposed to AICD protocol described in Example 19 and this Example 20. The results of this investigation demonstrate whether the peptide or peptide conjugates direct exposure to Th40 cells alters AICD susceptibility or if in vivo conditions involving the peptide is required. Treatment with CD40 targeting peptides, Au—NP CD40 targeting conjugates, and PLGA-NP CD40 targeting conjugates may show that the treatment with peptides influences cell death of Th40 cells. Further, treatment with the peptide or peptide conjugates may demonstrate that binding CD40 with a peptide or peptide conjugates may affect the levels of BCL-XL (B-cell lymphoma-extra large) and BCL2 (B-cell lymphoma 2), BIM (Bcl-2-like protein 11), BAK (Bcl-2 homologous antagonist killer), and cFLIP ((FADD-like IL-1β-converting enzyme)-inhibitory protein), each of which is associated with cell survival or death in Th40 cells.

### Example 17

CD40 targeting peptides, Au—NP CD40 targeting conjugates and PLGA-NP CD40 targeting conjugates may regulate CD40 mediated pathways in B cells and macrophages and provide an important tolerogenic effect. Accordingly, CD40 targeting peptides, Au—NP CD40 targeting conjugates and PLGA-NP CD40 targeting conjugates, nanoparticles alone, and/or anti-CD154 antibodies are administered at 3, 6, 9, and 12 weeks of age to mice subjects. The early time point is prior to auto-antibody (AA) development. Auto-antibodies in TID may be predictive of disease onset. While the role of AAs is not fully defined, the presence of AAs is associated with disease occurrence. At 15 weeks, serum is analyzed from each cohort for immunoglobin production and for specific diabetes associated AAs including but not limited to: insulin, GAD, IA-2, ZnT8, and others. In addition, total IgG and IgM production will be measured and treatments will be escalated to determine possible optimal treatment options.

The study demonstrates how the peptide alone, or with AuNPs of PLGA NPs may tolerize B cell AAs production. While targeting CD40 on B cells may impact AA production, it also may target B cell antigen presentation, and thus establish a novel mechanism for tolerance.

The subjects of Example 13, the BDC2.5 T cell clone and NOD mice are treated with CD40 binding peptides, Au—NP CD40 binding conjugates and PLGA-NP CD40 binding conjugates to compare administration techniques. Peptide functionalized NPs (Au NPs and PLGA-NPs) are injected via IV (intravenous), IP (intraperitoneal), or sub-cutaneous (SC), into mice at a concentration of 25 μg equivalent peptide and repeated for 16 weeks. At this point, approximately 80% of control subjects should be diabetic. Bare Au NPs and bare PLGA NPs without any peptides attached, bare peptides without NPs attached, and NPs with scrambled or irrelevant peptides will be used as controls. A positive anti-CD154 will also be used as a control.

B cells are isolated from the subject mice and analyzed for MHC-class I and MHC-class II molecules. Reduction in MHC-class I and MHC-class II indicates a reduced ability for antigen presentation. Moreover, CD80 and CD86 levels may be measured which may have a higher expression on activated B-cells.

B cells may also be used for antigen recall experiments. B cells from treated mice will be exposed to digested islets, for islet antigen source. B cells will also be exposed to chromogranin-A, the defined auto-antigen for the BDC2.5, TCR mice. Wild type T cells from untreated NOD mice and BDC2.5 T cell clones are labeled with tracking dye like CFSE. Cells will be exposed to B cells from each treatment cohort including CD40 binding peptides, Au—NP CD40 binding conjugates and PLGA-NP CD40 binding conjugates, and anti-CD154. T cell responses will be measured for proliferation and for cytokine production, both of which are measures of effector cell activity. Cell surface activation markers, including, CD25, CD154, CD69, CCR7, and others are examined on T cells. This example determines if CD40 binding peptide treatment alters B cell antigen presenting ability.

Moreover, naïve T cells are subjected to a similar protocol to determine if CD40 binding peptide treatment regulated B cells are capable of inducing pathogenic effector cells. In addition, B cells will be treated ex-vivo with CD40 binding peptides, Au—NP CD40 binding conjugates and PLGA-NP CD40 binding conjugates, to determine effects and dose escalated if necessary to determine dosing ranges and optimal doses for effects.

### Example 18

B cells are isolated from long-term treated mice of Example 20, and those B cells are characterized for their demonstrated regulatory capacity. B cells are examined for IL-10, TGFβ, and other cytokines associated with Bregs (B regulatory cells). B cells isolated from mice treated with CD40 binding peptide, Au—NP CD40 binding conjugates and PLGA-NP CD40 binding conjugates, are transferred to 3, 6, and 9 week old NOD mice that are monitored for hyperglycemia. This example will demonstrate whether disease is prevented by B cell transfers and whether the CD40 binding peptide increases Breg levels in subjects.

### Example 19

Macrophages are isolated from long-term treated mice of Example 20, and characterized for M1 versus M2 characteristics. Cytokine and chemokine production is analyzed from treated versus untreated mice. Because chemokines are responsible for chemo-attraction, tolerant macrophages may demonstrate different chemokine production, profiles, and patterns than macrophages during the disease state.

Adoptive transfer of M2 classified macrophages may be proven therapeutic in NOD mice. Therefore, isolated total macrophages from CD40 binding peptide treated, Au—NP CD40 binding conjugate treated, and PLGA-NP CD40 binding conjugate treated mice are transferred to mice of 6, 9, 12, and 16 week old mice.

CD40 binding peptide treatment may influence M2 tolerogenic macrophage development. If so, this finding may demonstrate and constitute a new tolerance mechanism in TID.

### Example 20

NOD mice that are 3 weeks of age are treated with CD40 binding peptides, Au—NP CD40 binding conjugates, and PLGA-NP CD40 binding conjugates. Control subjects are treated with NPs alone and a scrambled peptide. In these mice, insulitis has not occurred, so auto-antigen levels are at baseline levels. Treatment of mice will be performed via weekly injections (route of administration being either i.v. or subcutaneous). Pancreata and beta cells are isolated from mice treated for 3, 6, and 12 weeks. T cells and thymocytes are isolated from 3 week old mice. Thymocytes will be sorted to obtain mature T cells, CD4+ and CD8+. T cells are also isolated from spleen and regional lymph nodes and any potential Tregs are removed by negative sorting. APCs are isolated and exposed to beta cells from treated mice and beta cells from untreated mice are used as controls. T cell subsets are labelled with tracking dye, such as CFSE. T cell subsets are exposed to APCs and islets.

This example may demonstrate that T cell proliferation will indicate the presence of beta cell auto-antigens. T cells from NOD mice will likely respond to untreated islets. The example may demonstrate how cells from treated subjects respond, including whether treatment with CD40 binding peptide reduces auto-antigen production.

### Example 21

NOD mice that are 3 weeks of age are treated with CD40 binding peptides, Au—NP CD40 binding conjugates, and PLGA-NP CD40 binding conjugates. Control subjects are treated with NPs alone and a scrambled peptide. In these mice, insulitis has not occurred, so auto-antigen levels are at baseline levels. Treatment of mice will be performed via weekly injections (route of administration being either i.v. or subcutaneous).

T cells are isolated from NOD mice at 6, 9, 12, and 18 weeks. The mice of 18 weeks are considered to be at the age of diabetes onset and T cell breach of tolerance has occurred. T cells are labeled with tracker dye and exposed to APC+ treated and untreated islets.

This example may demonstrate if islets from CD40 binding peptide treated cohorts produce A-Ags (Auto-antigens). If T cells do not respond, it may be concluded that the protected islets produce less A-Ags.

### Example 22

Young NOD.scid mice are treated with CD40 binding peptides, Au—NP CD40 binding conjugates, and PLGA-NP CD40 binding conjugates. T cells are isolated from NOD mice at 3, 6, 9, 12, and 18 weeks of age. T cells are also isolated from spleen and regional lymph nodes and any potential Tregs are removed by negative sorting and remaining cells are transferred to treated NOD.scid recipients. Mice are then monitored over 12 weeks for diabetes development. In some instances these mice develop diabetes after 2 weeks; however, some mice are delayed in their development of diabetes for six weeks or more, so allowing for development for 12 weeks will ensure ample time for susceptible mice to develop disease.

The results of this example may demonstrate that in the instances where mice do not develop diabetes, the peptide treatment may be protective against beta cell auto-antigen production. Moreover, observations regarding diabetic disease development and transfer due to age of mice involved may demonstrate that the diabetic disease preventative benefits are time sensitive. For example, older mice receiving T cells from younger mice may not transfer disease. An additional example may be that younger mice receiving T cells from older mice may develop disease because older mice have a greater number of auto-aggressive (intolerant) T cells; however, if the antigen to which those T cells respond is not available, due to protection from the treatments, then diabetic disease state should not develop.

### Example 23

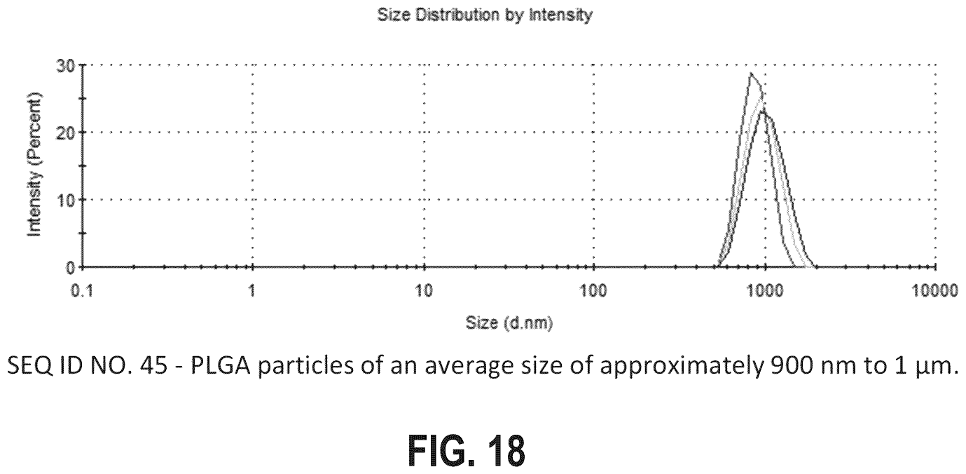

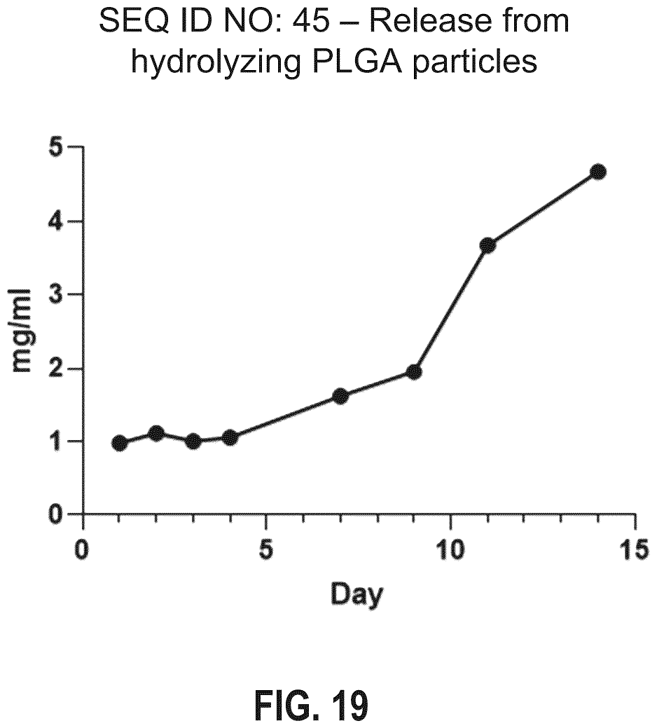

Aliquots of PLGA nanoparticles of SEQ ID NO: 45, with an average size of approximately 900 nm to 1 μm as shown in FIG. 18 were covered and placed in an environment at 37° C. At periodic points, remaining particles were pelleted by centrifugation and free peptide in the supernatant was measured by Bradford Protein Assay. The results are shown in the graph of FIG. 19. At day 14, there were still remaining particles, indicating that there may still be amounts of peptide to be released in this in-vitro setting.

### Example 24

A 0.4% w/v solution of Vitamin E-TPGS is prepared. This is performed by weighing out 4 g of Vitamin E-TPGS and adding to 1 liter sterile, deionized water in a Pyrex bottle. A stir bar is added, the bottled is capped, and allowed to stir overnight to dissolve.

Next, a 50 mg/ml solution of PLGA is prepared. This is done by weighing out 1 g of PLGA and dissolving in 20 ml of ethyl acetate. This is allowed to dissolved as much as possible by gentle swirling. If not fully dissolved, i.e. small particles are still seen, sonication with a 130 W sonicator set to 70% amplitude, may be utilized. Approximately, 5-10 bursts of 4-5 seconds each may be useful.

A portion of the 4% Vitamin E TPGS is diluted to 0.3% in a beaker. This solution of 0.3% Vitamin E-TPGS is placed on a magnetic stirrer, or the like, and stirred at approximately 360 rpm. This beaker should preferably be placed in a sterile environment to maintain sterility.

Next, 1 g of a selected peptide is combined with 12 ml PBS in a vial and allowed to dissolve. The vial is capped, then gently turned it on its side and slowly roll the vial. Stand the vial up again. The vial should continue to be rolled intermittently. The selected peptide will dissolve completely in about 10-15 minutes. A sterile cell scraper may be used to retrieve peptide from the walls of the vial.

40 borosilicate vials are prepared. 20 of them are placed on ice and 20 placed at room temperature.

A VCX 130 W Ultrasonic Processor (or similar), fitted with a tip for small volumes, is readied and set to 70% amplitude.

To the borosilicate vials on ice, 1 ml of the dissolved PLGA is added. 2 ml 0.4% Vitamin E-TPGS is added to each of the vials that are at room temperature.

Next, on ice, approximately 630 μl peptide is added to one of the vials of PLGA. With 2-3 second bursts, the contents are sonicated, about 8-10 times. This should result in a milky, homogenous emulsion. This may be repeated with a few sonication bursts if not completely homogenous.

Next, the emulsion is immediately transferred via a sterile polypropylene transfer pipet dropwise, while vortexing, to a vial of 0.4% Vitamin E TPGS. Care is taken to deliver each drop in the center of the vial and not to touch the sides. When the whole content is added, continue vortexing for about 15 more seconds. The vial is returned to ice and then immediately sonicated and followed by a short vortex. This sonication and vortexing is repeated twice, for a total of 3 sonications and vortexing.

A sterile polypropylene transfer pipet is used to aspirate 1-2 ml of 0.3% Vitamin E TPGS from the beaker that is stirring. This 1-2 ml of 0.3% Vitamin E TPGS is added to the emulsion in the borosilicate vial to thin the emulsion. The contents of the vial are then poured into a clean beaker. This process may be repeated to retrieve as much as possible from the vial into the beaker.

The above steps are repeated for each of the remaining vials. It is preferred that this is done as rapidly as possible so as little delay as possible.

Once all of the vials are processed, the beaker containing the pooled vials is allowed to sit for 3 hours, uncovered. This may help age/harden the particles and allow the ethyl acetate to evaporate.

The contents of the beaker are then allocated into Oak Ridge, or similar, polysulfone centrifuge tubes with sealing caps. The vials should with the same or closely similar amounts in preparation for centrifuge. Centrifuge at 17,500×g (11,000 rpm in an SA-600 rotor; 12,000 rpm in a SS-34 rotor), 4° C., for 15 minutes to pellet the nanoparticles. Depending on the rotor capacity, this step may need to be done twice to pellet all the material. (NOTE: The vials must be filled to at least 80% capacity or their integrity will be compromised during the high speed.)

From one of the vials, 1 ml of the supernatant is retrieved for later protein measurement. This will determine the amount of free peptide that has not been encapsulated.

Next, the supernatant is decanted from all the vials and resuspend the particles from half of the vials in 10 ml sterile water. Likewise, resuspend the other half of the vials in 10 ml water. The particles are now pooled into two vials. Next, on ice, the particles are sonicated with approximately 8-10 bursts, for approximately 2-3 seconds each.

Next, water is used to rinse the previous vials to retrieve as much as possible of the particles and add it to the two vials with sonicated nanoparticles. Fill the vials to the top with water and balance them for centrifugation. Centrifuge at 17,500×g (11,000 rpm in an SA-600 rotor; 12,000 rpm in a SS-34 rotor), 4° C., for 15 minutes to pellet the nanoparticles. The process of decanting, resuspension, and sonication is repeated again.

The washed particles are then resuspended in 40 ml sterile PBS. A cryoprotectant, such as 0.5 g Trehalose, may be added and allow to dissolve. (Trehalose should be kept at a PLGA: Trehalose ratio of 2:1.)

The final suspension may be sonicated, as above, on ice, to assure dispersion of the particles. 1 ml of the resuspended particles may be desired to be used to measure the of encapsulated peptide. The remaining resuspended particles are aliquoted into sterile vials; 4 ml per vial may be desired. Freeze the vials at −80° C.

The above described process should be done as aseptically as possible, using autoclaved glassware and sterile solutions.

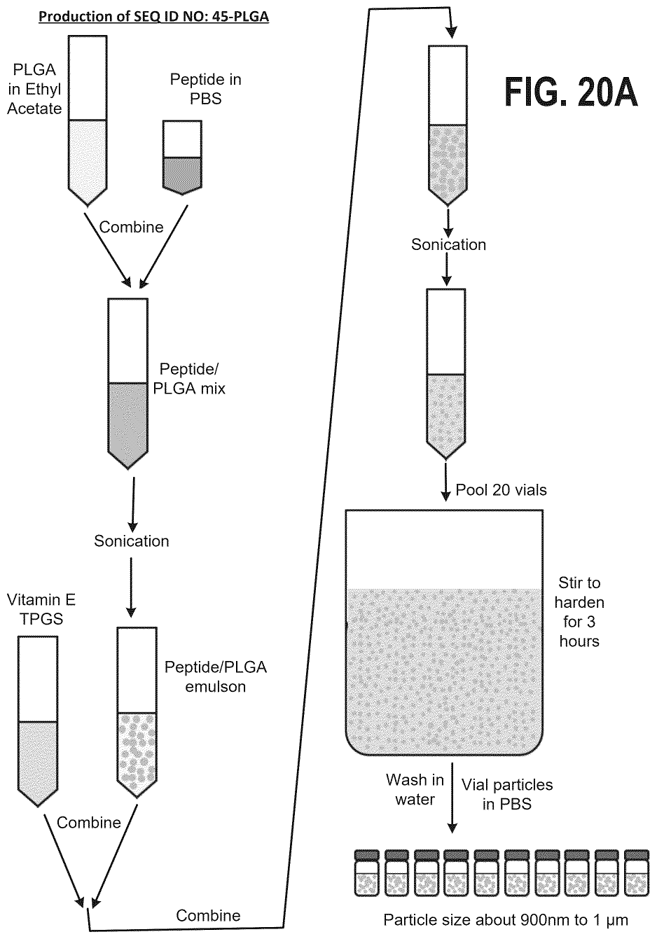

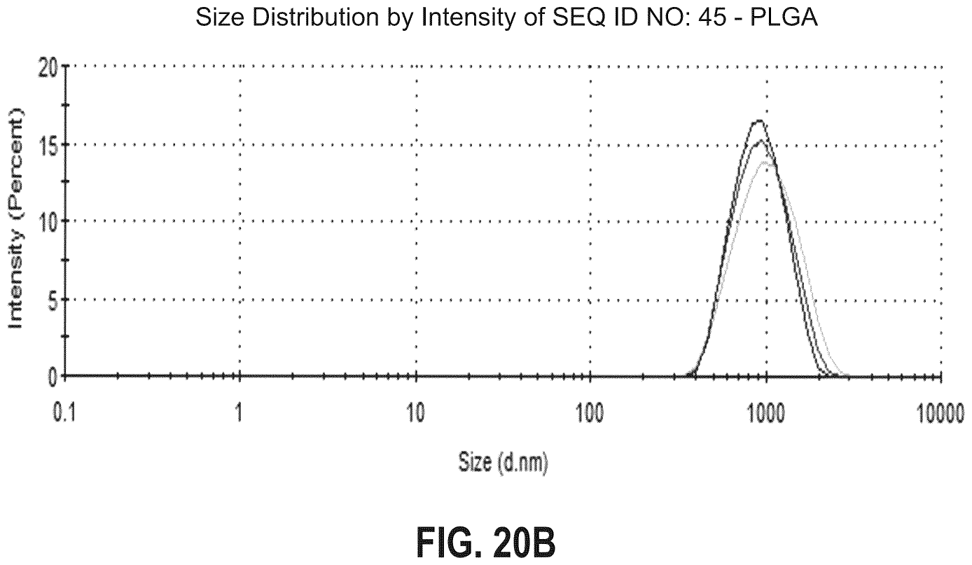

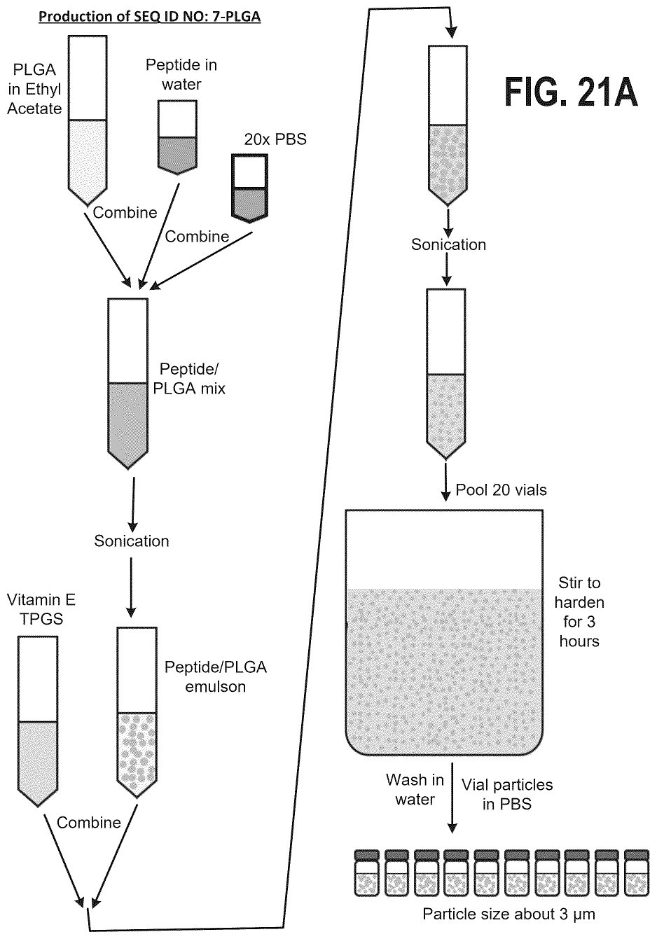

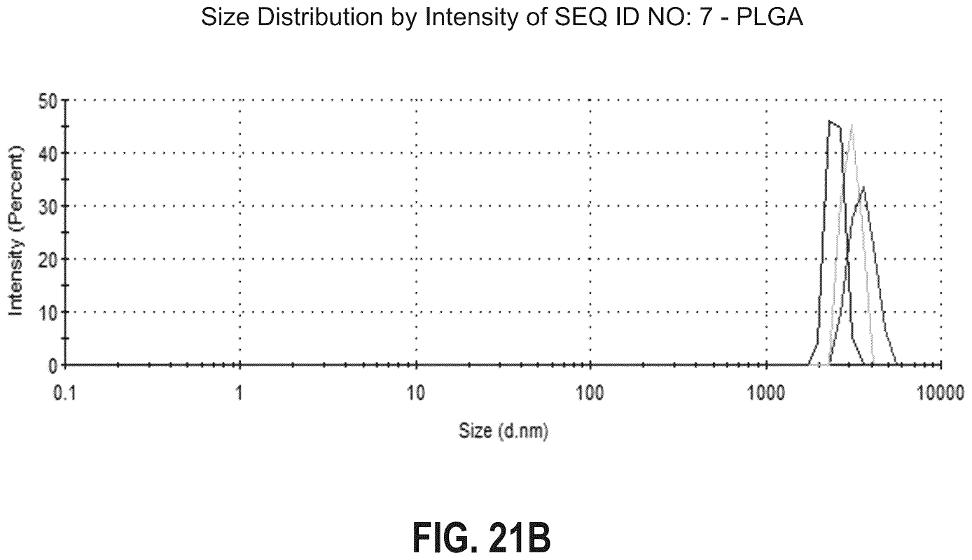

This process is disclosed and described in FIG. 20A and FIG. 21A.

FIG. 20A is a schematic diagram that shows the process that is described above, in this Example 24.

FIG. 21A is a schematic diagram that shows that a 20× concentrated PBS solution may be utilized in forming the peptide/PLGA mix. Note, that in FIG. 21B, below, the particles were approximately 3000 nm in diameter.

FIG. 20B provides a graph of the Dynamic Light Scattering measurement of a PLGA nanoparticle and a peptide of SEQ ID NO: 45.

FIG. 21B provides a graph of the Dynamic Light Scattering measurement of a PLGA nanoparticle and a peptide of SEQ ID NO: 7.

The various features, processes, and implementations described above may be used independently of one another or may be combined in various ways. All possible combinations and sub-combinations are intended to fall within the scope of this disclosure. In addition, certain method or process blocks may be omitted in some implementations. While certain example implementations have been described, these implementations have been presented by way of example only and are not intended to limit the scope of the inventions disclosed herein. Thus, nothing in the foregoing description is intended to imply that any particular feature, characteristic, step, module, or block is necessary or indispensable. Indeed, the novel methods and compositions described herein may be embodied in a variety of other forms; furthermore, various omissions, substitutions, and changes in the form of the methods and systems described herein may be made without departing from the spirit of the inventions disclosed herein. The accompanying claims and their equivalents are intended to cover such forms and modifications as would fall within the scope and spirit of certain of the inventions disclosed herein.

### REFERENCES

Bajorath J, Chalupny N J, Marken J S, et al. Identification of residues on CD40 and its ligand which are critical for the receptor-ligand interaction. Biochemistry. 1995; 34:1833-1844.
Bajorath J, Marken J S, Chalupny N J, et al. Analysis of gp39/CD40 interactions using molecular models and site-directed mutagenesis. Biochemistry. 1995; 34:9884-9892.
Balasa B, Krahl T, Patstone G, et al. CD40 ligand-CD40 interactions are necessary for the initiation
of insulitis and diabetes in nonobese diabetic mice. J Immunol. 1997; 159:4620-4627. [PubMed: 9379064]
Bosmans L A, Bosch L, Kusters P J H, Lutgens E, Seijkens T T P. The CD40-CD40L Dyad as Immunotherapeutic Target in Cardiovascular Disease. J Cardiovasc Transl Res. 2020 Mar. 28. doi: 10.1007/s12265-020-09994-3.
Boumpas D T, Furie R, Manzi S, et al. A short course of BG9588 (anti-CD40 ligand antibody) improves serologic activity and decreases hematuria in patients with proliferative lupus glomerulonephritis. Arthritis Rheum. 2003; 48:719-727.
Danhier F, Ansorena E, Silva J M, Coco R, Breton A, Préat V. PLGA-based nanoparticles: An overview of biomedical applications. Journal of Controlled Release. Volume 161, Issue 2, 20 Jul. 2012, Pages 505-522.
Davis J C Jr, Totoritis M C, Rosenberg J, Sklenar T A, Wofsy D. Phase I clinical trial of a monoclonal antibody against CD40-ligand (IDEC-131) in patients with systemic lupus erythematosus. J Rheumatol. 2001; 28:95-101.
Deambrosis I, Lamorte S, Giaretta F, et al. Inhibition of CD40-CD154 costimulatory pathway by a cyclic peptide targeting CD154. J Mol Med (Berl). 2009; 87:181-197.
Durie F H, Fava R A, Foy T M, Aruffo A, Ledbetter J A, Noelle R J. Prevention of collagen-induced arthritis with an antibody to gp39, the ligand for CD40. Science. 1993; 261:1328-1330.
Girvin A M, Dal Canto M C, Miller S D. CD40/CD40L Interaction is Essential for the Induction of EAE in the Absence of CD28-Mediated Co-stimulation. Journal of Autoimmunity, Volume 18, Issue 2, March 2002, Pages 83-94.
Howard L M, Miga A J, Vanderlugt C L, et al. Mechanisms of immunotherapeutic intervention by anti-CD40L (CD154) antibody in an animal model of multiple sclerosis. J Clin Invest. 1999; 103:281-290.
Kaur M, Reed E, Sartor O, Dahut W, Figg W D. Suramin's development: what did we learn? Invest New Drugs. 2002; 20:209-219.
Kitagawa M, Goto D, Mamura M, et al. Identification of three novel peptides that inhibit CD40-CD154 interaction. Mod Rheumatol. 2005; 15:423-426.
Kobata T, Azuma M, Yagita H, Okumura K. Role of costimulatory molecules in autoimmunity. Rev Immunogenet. 2000; 2:74-80.
Margolles-Clark E, Umland O, Kenyon N S, Ricordi C, Buchwald P. Small-molecule costimulatory blockade: organic dye inhibitors of the CD40-CD154 interaction. J Mol Med (Berl). 2009; 87:1133-1143.
Margolles-Clark E, Jacques-Silva M C, Ganesan L, et al. Suramin inhibits the CD40-CD154 costimulatory interaction: a possible mechanism for immunosuppressive effects. Biochem Pharmacol. 2009; 77:1236-1245.
Munroe M E, Bishop G A. A costimulatory function for T cell CD40. J Immunol. 2007; 178:671-682.
Quezada S A, Eckert M, Adeyi O A, Schned A R, Noelle R J, Burns C M. Distinct mechanisms of action of anti-CD154 in early versus late treatment of murine lupus nephritis. Arthritis Rheum. 2003; 48:2541-2554.
Sidiropoulos P I, Boumpas D T. Lessons learned from anti-CD40L treatment in systemic lupus erythematosus patients. Lupus. 2004; 13:391-397.
Toubi E, Shoenfeld Y. The role of CD40-CD154 interactions in autoimmunity and the benefit of disrupting this pathway. Autoimmunity. 2004; 37:457-464.
Vaitaitis G M, Poulin M, Sanderson R J, Haskins K, Wagner D H Jr. Cutting edge: CD40-induced expression of recombination activating gene (RAG) 1 and RAG2: a mechanism for the generation of autoaggressive T cells in the periphery. J Immunol. 2003; 170:3455-3459.
Vaitaitis G M, Wagner D H Jr. High distribution of CD40 and TRAF2 in Th40 T cell rafts leads to preferential survival of this auto-aggressive population in autoimmunity. PLOS One. 2008; 3: e2076.
Vaitaitis G M, Yussman M G, Waid D M, Wagner D H. Th40 cells (CD4+CD40+ Tcells) drive a more severe form of Experimental Autoimmune Encephalomyelitis than conventional CD4 Tcells. PLOS One. 2017 Feb. 13; 12(2):e0172037. doi: 10.1371/journal.pone.0172037.eCollection 2017.
Vaitaitis G M, Rihanek M, Alkanani A K, Waid D M, Gottlieb P A, Wagner D H; Type 1 Diabetes TrialNet Study Group. Biomarker discovery in pre-Type 1 Diabetes; Th40 cells as a predictive risk factor. J Clin Endocrinol Metab. 2019 May 7; 104(9):4127-42. doi: 10.1210/jc.2019-00364.
Vaitaitis G M, Yussman M G, Wagner D H. A CD40 targeting peptide prevents severe symptoms in experimental autoimmune encephalomyelitis. J Neuroimmun., Volume 332, 15 Jul. 2019, Pages 8-15.
Waid D M, Vaitaitis G M, Wagner D H Jr. Peripheral CD410CD40+ auto-aggressive T cell expansion during insulin-dependent diabetes mellitus. European J Immunol. 2004; 34:1488-1497.
Waid D M, Wagner R J, Putnam A, Vaitaitis G M, Pennock N D, Calverley D C, Gottlieb P, Wagner D H. A unique T cell subset described as CD4loCD40+ T cells (TCD40) in human type 1 diabetes. Clin Immunol. 2007 August; 124(2): 138-48. Epub 2007 Jun. 8.
Waid D M, Schreiner T, Vaitaitis G, Carter J R, Corboy J R, Wagner D H Jr. Defining a new biomarker for the autoimmune component of Multiple Sclerosis: Th40 cells. J Neuroimmunol. 2014 May 15; 270(1-2):75-85. doi: 10.1016/j.jneuroim.2014.03.009. Epub 2014 Mar. 15.
Wang X, Huang W, Mihara M, Sinha J, Davidson A. Mechanism of action of combined short-term CTLA4Ig and anti-CD40 ligand in murine systemic lupus erythematosus. J Immunol. 2002; 168:2046-2053.
Yu S, Medling B, Yagita H, Braley-Mullen H. Characteristics of inflammatory cells in spontaneous autoimmune thyroiditis of NOD.H-2 h4 mice. J Autoimmun. 2001; 16:37-46.

1.-117. (canceled)

118. A method of treating alopecia in a subject comprising administering to the subject a therapeutically effective amount of a peptide comprising the amino acid sequence of SEQ ID NO: 7.

119. The method of claim 118, wherein the alopecia is an autoimmune related or induced alopecia.

120. The method of claim 118, wherein the peptide comprises a chemical modification.

121. The method of claim 120, wherein the peptide is pegylated or glycosylated.

122. The method of claim 120, wherein the peptide is modified by acetylation on the N-terminus and/or amidation on the C-terminus.

123. The method of claim 118, wherein the peptide binds to a CD40 protein with a Kd of less than 10-6.

124. The method of claim 118, wherein the peptide affects an interaction of CD40 with a CD40 ligand.

125. The method of claim 118, wherein the peptide affects the interaction of CD40 and CD154.

126. The method of claim 118, wherein the peptide inhibits the binding of CD40 to CD154.

127. The method of claim 118, wherein the peptide prevents expansion of Th40 cells.

128. The method of claim 118, wherein the peptide does not elicit antibody production in the subject.

129. The method of claim 118, wherein the peptide reduces hair loss in the subject.

130. The method of claim 118, wherein the peptide is 15 amino acid long.

131. The method of claim 118, wherein the peptide consists of the amino acid sequence of SEQ ID NO: 7.


SEQUENCE LISTING (50 sequences)

SEQ ID NO: 1 - protein, 260 aa, Mus musculus
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 2 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 3 - protein, 4 aa, Mus musculus
KGYY

SEQ ID NO: 4 - protein, 6 aa, synthetic construct
AKKGYY

SEQ ID NO: 5 - protein, 8 aa, synthetic construct
AKKGYYTM

SEQ ID NO: 6 - protein, 8 aa, synthetic construct
AEKGYYTM

SEQ ID NO: 7 - protein, 15 aa, synthetic construct
VLQWAKKGYYTMKSK

SEQ ID NO: 8 - protein, 15 aa, synthetic construct
VLQWAEKGYYTMSNN

SEQ ID NO: 9 - protein, 24 aa, synthetic construct
NAASVLQWAKKGYYTMKSNLVMLE

SEQ ID NO: 10 - protein, 17 aa, synthetic construct
ISQAVHAAHAEINEAGR

SEQ ID NO: 11 - protein, 15 aa, synthetic construct
GLQWAKKGYYTMKSN

SEQ ID NO: 12 - protein, 15 aa, synthetic construct
VGQWAKKGYYTMKSN

SEQ ID NO: 13 - protein, 15 aa, synthetic construct
VLGWAKKGYYTMKSN

SEQ ID NO: 14 - protein, 15 aa, synthetic construct
VLQGAKKGYYTMKSN

SEQ ID NO: 15 - protein, 15 aa, synthetic construct
VLQWGKKGYYTMKSN

SEQ ID NO: 16 - protein, 15 aa, synthetic construct
VLQWAGKGYYTMKSN

SEQ ID NO: 17 - protein, 15 aa, synthetic construct
VLQWAKGGYYTMKSN

SEQ ID NO: 18 - protein, 15 aa, synthetic construct
VLQWAKKGGYTMKSN

SEQ ID NO: 19 - protein, 15 aa, synthetic construct
VLQWAKKGYGTMKSN

SEQ ID NO: 20 - protein, 15 aa, synthetic construct
VLQWAKKGYYGMKSN

SEQ ID NO: 21 - protein, 15 aa, synthetic construct
VLQWAKKGYYTGKSN

SEQ ID NO: 22 - protein, 17 aa, synthetic construct
ISQAVHAAHAEINEAGR

SEQ ID NO: 23 - protein, 15 aa, synthetic construct
YVQGKANLKSKLMYT

SEQ ID NO: 24 - protein, 10 aa, synthetic construct
WAKKGYYTMK

SEQ ID NO: 25 - protein, 13 aa, synthetic construct
VLQWAKKGYYTMK

SEQ ID NO: 26 - protein, 24 aa, synthetic construct
AASVLQWAKKGYYTMKSNLVVLEN

SEQ ID NO: 27 - protein, 6 aa, synthetic construct
KGYYTM

SEQ ID NO: 28 - protein, 6 aa, synthetic construct
AEKGYY

SEQ ID NO: 29 - protein, 6 aa, synthetic construct
KKGYYT

SEQ ID NO: 30 - protein, 6 aa, synthetic construct
AKGYYT

SEQ ID NO: 31 - protein, 6 aa, synthetic construct
VKKGYY

SEQ ID NO: 32 - protein, 15 aa, synthetic construct
VLRWAPKGYYTISSN

SEQ ID NO: 33 - protein, 15 aa, synthetic construct
VLQWAPKGYYTISSN

SEQ ID NO: 34 - protein, 15 aa, synthetic construct
VLQWAQKGYYTISNN

SEQ ID NO: 35 - protein, 11 aa, synthetic construct
YGRKKRRQRRR

SEQ ID NO: 36 - protein, 11 aa, synthetic construct
  VARIANT 1: note = Any amino acid other than Tyr
XGRKKRRQRRR

SEQ ID NO: 37 - protein, 10 aa, synthetic construct
GRKKRRQRRR

SEQ ID NO: 38 - protein, 260 aa, unidentified
  source 1..260: note = Canis familiaris
MIETYSQTAPRSVATGPPVSMKIFMYLLTVFLITQMIGSALFAVYLHRRLDKIEDERNLYEDFVFMKTLQKCNKGEGSLSLLNCEEIKSQFEAFLKEIMLNNEMKKEENIAMQKGDQDPRIAAHVISEASSNPASVLRWAPKGYYTISSNLVSLENGKQLAVKRQGLYYVYAQVTFCSNRAASSQAPFVASLCLHSPSGTERVLLRAASSRGSSKPCGQQSIHLGGVFELHPGASVFVNVTDPSQVSHGTGFTSFGLLKL

SEQ ID NO: 39 - protein, 260 aa, Felis catus
MIETYSQTAPRSVAPGPPVSMKIFMYLLTVFLITQMIGSALFAVYLHRRLDKIEDERNLYEDFVFMKTLQKCNKGEGALSLLNCEEIKSRFEAFLKEIMLNKETKKEKNVAMQKGDQDPRVAAHVISEASSSTASVLQWAPKGYYTISSNLVTLENGKQLAVKRQGLYYIYAQVTFCSNREASSQAPFIASLCLHSPSGSERVLLRAANARSSSKPCGQQSIHLGGVFELHPGASVFVNVTDPSQVSHGTGFTSFGLLKL

SEQ ID NO: 40 - protein, 261 aa, Equus caballus
MIETYSQPSPRSVATGPPVSMKIFMYLLTVFLITQMIVSALFAVYLHRRLDKIEDERNLHEDFVFMKTIQRCNKGEGPLSLLNCEEIRSQFEGFVKDIMLNEEVKKKGENFEMQKGDQEPQIAAHVISEASSKTASVLQWAQKGYYTISNNLVTLENGKQLAVKRQGLYYIYAQVTFCSNREASGQAPFIASLCLRSVSGSERILLRAANTHSSSKPCGQQSIHLGGVFELQPGASVFVNVTDPSQVSHGTGFTSFGLLKL

SEQ ID NO: 41 - protein, 6 aa, synthetic construct
KKGYYT

SEQ ID NO: 42 - protein, 6 aa, synthetic construct
EKGYYT

SEQ ID NO: 43 - protein, 6 aa, synthetic construct
PKGYYT

SEQ ID NO: 44 - protein, 6 aa, synthetic construct
QKGYYT

SEQ ID NO: 45 - protein, 15 aa, synthetic construct
VLRWAPKGYYTISSN

SEQ ID NO: 46 - protein, 15 aa, synthetic construct
VLQWAPKGYYTISSN

SEQ ID NO: 47 - protein, 15 aa, synthetic construct
VLQWAQKGYYTISNN

SEQ ID NO: 48 - protein, 24 aa, synthetic construct
NAASVLQWAKKGYYTMKSNLVMLE

SEQ ID NO: 49 - protein, 6 aa, synthetic construct
APKGYY

SEQ ID NO: 50 - protein, 15 aa, synthetic construct
YKNVKQMAYWLTGKS